SEQUENCE LISTING (158 sequences)

SEQ ID NO: 1 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 2 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 3 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 4 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 5 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 6 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 7 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgaagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 8 - protein, 323 aa, synthetic construct
MASMDKVFSGYYARQKLLERSDNPFSKGIAYVEGKFVLPSDARIPLLDPGFTKSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 9 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgtttagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 10 - protein, 323 aa, synthetic construct
MASMDKVFSGYYARQKLLERSDNPFSKGIAYVEGKFVLPSDARIPLLDPGFTFSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 11 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacggagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 12 - protein, 323 aa, synthetic construct
MASMDKVFSGYYARQKLLERSDNPFSKGIAYVEGKFVLPSDARIPLLDPGFTESWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 13 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcatagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 14 - protein, 323 aa, synthetic construct
MASMDKVFSGYYARQKLLERSDNPFSKGIAYVEGKFVLPSDARIPLLDPGFTHSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 15 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcgggattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 16 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVGIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 17 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcgagattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 18 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVEIIVTRGLTGVRGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 19 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 20 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 21 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtggggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 22 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQWGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 23 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 24 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 25 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 26 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 27 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagacgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 28 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQTGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 29 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcctgttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 30 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQWGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 31 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtggggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 32 - protein, 323 aa, synthetic construct; identical to SEQ ID NO: 2

SEQ ID NO: 33 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 34 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 35 - DNA, 972 bp, synthetic construct; identical to SEQ ID NO: 25
  source 1..972: mol_type = other DNA

SEQ ID NO: 36 - protein, 323 aa, synthetic construct; identical to SEQ ID NO: 26

SEQ ID NO: 37 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 38 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 39 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 40 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 41 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 42 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 43 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 44 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 45 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 46 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 47 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 48 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 49 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 50 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 51 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 52 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 53 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 54 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 55 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 56 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 57 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 58 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 59 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 60 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 61 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgcagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 62 - protein, 323 aa, synthetic construct
MASMDKVFSGYYARQKLLERSDNPFSKGIAYVEGKFVLPSDARIPLLDPGFTQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 63 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 64 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 65 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 66 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 67 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 68 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 69 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 70 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 71 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 72 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 73 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 74 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 75 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 76 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 77 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 78 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 79 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 80 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 81 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 82 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 83 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 84 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 85 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 86 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 87 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 88 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 89 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 90 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 91 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 92 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 93 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggttttacgactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 94 - protein, 323 aa, synthetic construct
MASMDKVFSGYYARQKLLERSDNPFSKGIAYVEGKFVLPSDARIPLLDPGFTTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 95 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 96 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 97 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 98 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 99 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 100 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 101 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 102 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 103 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 104 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 105 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 106 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 107 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 108 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 109 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 110 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 111 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 112 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 113 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 114 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 115 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 116 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 117 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 118 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 119 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 120 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 121 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 122 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 123 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 124 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 125 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttaccagagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 126 - protein, 323 aa, synthetic construct
MASMDKVFSGYYARQKLLERSDNPFSKGIAYVEGKFVLPSDARIPLLDPGFYQSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 127 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 128 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 129 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 130 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 131 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 132 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 133 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 134 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 135 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 136 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 137 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 138 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 139 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 140 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 141 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttcgcggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 142 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVRGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 143 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 144 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 145 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 146 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 147 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 148 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 149 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatatctggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 150 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYIWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 151 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 152 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 153 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcaggcgggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 154 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQAGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 155 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcaacttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 156 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGNFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

SEQ ID NO: 157 - DNA, 972 bp, synthetic construct
  source 1..972: mol_type = other DNA
atggcaagcatggataaagtcttcagcggctattacgcgcgtcaaaaactgctggaacgctctgataacccgtttagcaaaggcatcgcgtacgttgaaggtaaatttgttctgccgagcgacgcacgtattccgctgctggacccgggtttttacactagctgggcgacctacactaccattagcgtttgggacggccgctttttccgtctggacgatcatctgaaacgtctgctggaatcctgcgacaaatctcgcctgaaatttccgctggcactgagcagcgttcgcaaaatcctggcggaaatggttgcgaaatctggtattcgcgatgcgttggtcaacattattgttacccgcggtctgaccggcgttctgggtcgccaaccggaagacctgccgaacccgaacatctacctgctggttctgccgtatcagtggctgatggcaccggaaaaacaacgtcacggcggccatgcaattattacccgtaccgttcgtcgtaccccgccgggcgcattcgatccgaccattaaaaactttcagtctggcgatctgcgcaaaggcattttcgaagcagcggatcgcggcgcagaatatccgtttctgaccgacggcgatacccacctgaccgaaggtagcggttttaacatcgtcctggtcaaaaacggcatcatctacaccccggatcgcggcgttctggaaggtattacccgtcagagcgttattgacgttgcacgcgcaaacagtattgatacccgcctggaagtcgtcccggttgaagcggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcaactaa

SEQ ID NO: 158 - protein, 323 aa, synthetic construct
MASMDKVFSGYYARQKLLERSDNPFSKGIAYVEGKFVLPSDARIPLLDPGFYTSWATYTTISVWDGRFFRLDDHLKRLLESCDKSRLKFPLALSSVRKILAEMVAKSGIRDALVNIIVTRGLTGVLGRQPEDLPNPNIYLLVLPYQWLMAPEKQRHGGHAIITRTVRRTPPGAFDPTIKNFQSGDLRKGLFEAADRGAEYPFLTDGDTHLTEGSGFNIVLVKNGIIYTPDRGVLEGITRQSVIDVARANSIDTRLEVVPVEAAYHSDEIFMTSTAGGIMPITLLDGQPVNGGQVGPITKKIWDGYWEMHYNNAYSFPVDYGSN

(19) United States
(12) Patent Application Publication
(10) Pub. No.: US 2025/0188502 A1
(43) Pub. Date: Jun. 12, 2025

(54) BIOCATALYST AND METHOD FOR THE SYNTHESIS OF UBROGEPANT INTERMEDIATES

(71) Applicant: Enzymaster (Ningbo) Bio-Engineering Co., Ltd., Ningbo, Zhejiang (CN)

(72) Inventors: Haibin CHEN, Ningbo Zhejiang (CN); Hu HU, Ningbo Zhejiang (CN); Baoqin CAI, Ningbo Zhejiang (CN); Junpeng XU, Ningbo Zhejiang (CN); Sitong LIU, Ningbo Zhejiang (CN); Luping JIN, Ningbo Zhejiang (CN); Xinwei WU, Ningbo Zhejiang (CN); Qinyan CUI, Ningbo Zhejiang (CN); Chengxiao ZHANG, Ningbo Zhejiang (CN); Kailong ZHU, Ningbo Zhejiang (CN)

(73) Assignee: Enzymaster (Ningbo) Bio-Engineering Co., Ltd., Ningbo, Zhejiang (CN)

(21) Appl. No.: 18/845,491

(22) PCT Filed: Feb. 17, 2023
(86) PCT No.: PCT/CN2023/076973

§ 371 Date: Sep. 10, 2024

(30) Foreign Application Priority Data
Mar. 10, 2022 (CN) 202210240991.5

(51) Int. Cl. C12P 17/18; C12N 9/10

(52) U.S. Cl. CPC C12P 17/182; C12N 9/1096; C12Y 206/01

(57) ABSTRACT

This application provides engineered transaminase polypeptides for the synthesis of Ubrogepant intermediates with high stereoselectivity, high catalytic activity and good stability. This application also provides a reaction process for the asymmetric synthesis of Ubrogepant intermediates using the transaminase polypeptide.

The present invention relates to the field of bioengineering technology, and in particular to the application of an engineered transaminase polypeptide for the catalytic synthesis of Ubrogepant intermediates.

### BACKGROUND TECHNOLOGY

Migraine is a common primary headache condition characterized by unilateral moderate to severe throbbing headache and may be accompanied by nausea, vomiting, photophobia, etc. Currently, more than 10% of the world's population suffers from migraine, with approximately twice as many women as men. There are complex genetic causes for migraine, but it is not clear which genes are associated with the development of migraine, and the pathogenesis of migraine is not clearly established. Traditionally, migraine has been treated with trimethoprim drugs such as sumatriptan, zolmitriptan and almotriptan, which mainly act on 5HT1B/1D receptors, making them inappropriate for use in migraine patients with cardiovascular disease due to their inherent vasoconstrictor activity. Calcitonin gene-related peptide (CGRP) is a 37 amino acid neuropeptide with vasodilatory effects that acts at multiple sites and is involved in injury sensitization and sensitization of peripheral and central neurons in the trigeminal vascular system, which is relevant to the pathophysiology of migraine. CGRP receptor antagonism has now been shown to be an effective modality for migraine relief. Ubrogepant, which was approved for marketing by the FDA in 2019, is the first oral calcitonin gene-related peptide (CGRP) receptor antagonist approved by the FDA for the treatment of migraine. Ubrogepant relieves migraine symptoms by blocking the binding of CGRP to its receptor, acting in a new way with a completely different mechanism of action from that of the traditional tritans, and without constricting blood vessels, a problem with many existing migraine treatment drugs.

In the paper “Practical Asymmetric Synthesis of a Calcitonin Gene-Related Peptide (CGRP) Receptor Antagonist Ubrogepant” (Org. Process Res. Dev. 2017, 21, 1851′ 1858), Nobuyoshi Yasuda et al. disclosed a synthetic method for Ubrogepant (see FIG. 1). Ubrogepant contains 2 core structural fragments, one of which is a lactam containing 3 chiral centers (as shown in structural formula L2) and the other fragment is spiroic acid (as shown in structural formula A1), with the lactam being the most difficult to synthesize. To synthesize the key intermediate L2, Nobuyoshi Yasuda et al. first synthesized the compound shown as structural formula S1 (isopropyl 4-phenyl-2-(tert-butoxycarbonylamino)-5-oxohexanoate) and used S1 (theoretically containing four different isomers: ST1, ST2, SD1 and SD2) as the substrate in a 50% dimethyl sulfoxide (DMSO) condition for the dynamic kinetic reaction catalyzed by transaminase to obtain the lactam L1 with two chiral centers fixed. This transaminase-catalyzed reaction utilizes epimerization of S1 to prepare L1 with high chiral purity in one step. If transaminase is active only for the isomers ST1 and ST2, but not for SD1 or SD2 in the S1 substrate, ST1 and ST2 are converted to IT1 and IT2 by transaminase, respectively (the ester bonds in the structures of IT1 and IT2 can spontaneously break and then form a ring to give L1), and there will be no ID1 or ID2 in the products; meanwhile, under suitable reaction conditions, with the consumption of ST1 and ST2, the isomers SD1 and SD2 that fail to participate in the transaminase reaction can be spontaneously converted to ST1 and ST2 in situ, and the resulting ST1 and ST2 are then converted to IT1 and IT2 by transaminase. The key to this dynamic kinetic reaction is to develop a transaminase that is highly selective for the isomers ST1 and ST2 (i.e., active only for ST1 and ST2 but not for SD1 or SD2, in converting the target carbonyl group to an amino group), and only the R-configuration amino group is generated. This results in an extremely high chiral purity of the resulting lactam L1. In the transaminase reaction disclosed by Nobuyoshi Yasuda et al., the ratio of the sum of IT1 and IT2 to the sum of ID1 and ID2 in the product (i.e., diastereomeric ratio, dr for short) was up to 61:1; once intermediate L1 was obtained by the transaminase reaction, Nobuyoshi Yasuda et al. performed alkylation reaction and preparation of L2 using crystallization-induced diastereomeric transformation.

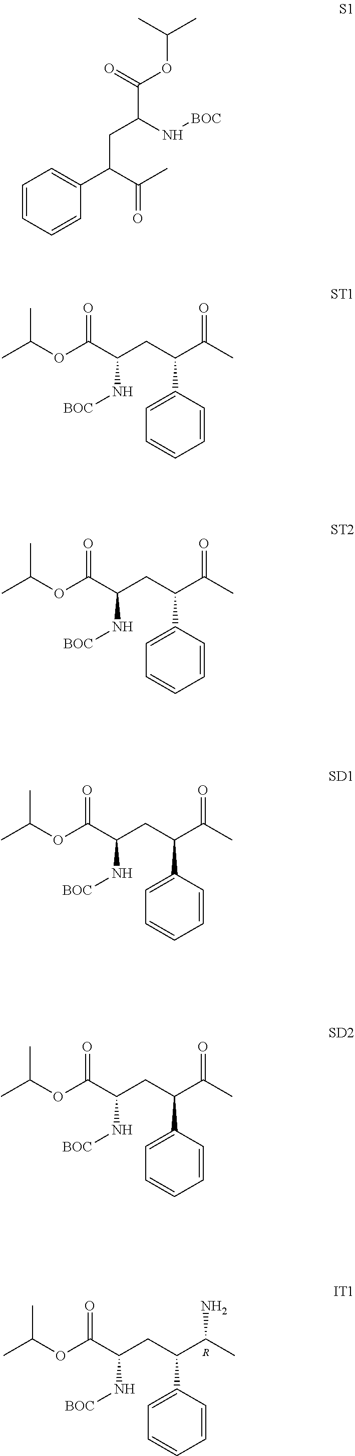

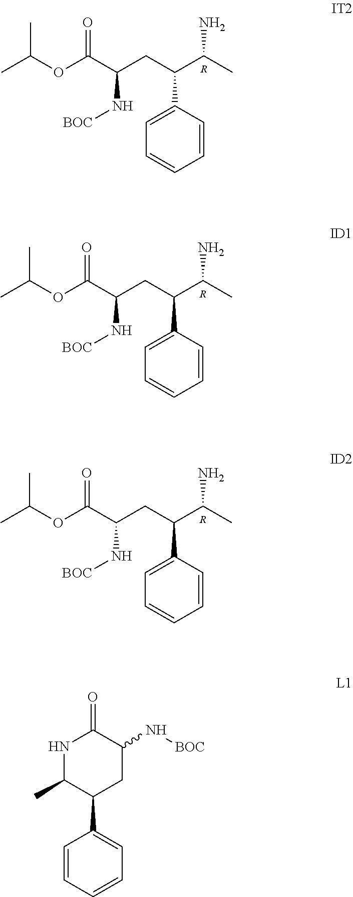

The present invention discloses an engineered transaminase with improved performance which is used in dynamic kinetic transaminase-catalyzed reactions for the synthesis of L1 and its analogs. The engineered transaminase provided by the present invention has better tolerance to the solvent used in the reaction, better selectivity, better activity and better thermal stability. Meanwhile, the present invention optimizes the transaminase reaction condition and post-treatment procedure, using a mixture of dimethyl sulfoxide (DMSO) and acetonitrile (ACN) as the reaction cosolvent. This greatly overcomes the defect of low solubility of substrate S1 when DMSO is used as a single cosolvent, and avoids the harm to enzyme activity from high concentration of acetonitrile which, when used as a single cosolvent, gives much higher solubility of the substrate S1. Moreover, cosolvents can be partially recycled in the post-treatment process, which is more economical and environmentally friendly.

### Content of the Invention

The present invention provides an engineered transaminase polypeptide with high stereoselectivity, high catalytic activity and good stability, capable of asymmetrically synthesizing chiral amines, in particular, asymmetrically synthesizing the intermediate L1 of Ubrogepant. Also provided are genes encoding engineered transaminase polypeptides, recombinant expression vectors containing the genes, engineered strains and efficient preparation methods thereof. The reaction process and product purification process for the asymmetric synthesis of L1 using the engineered transaminase peptide are also provided.

A first aspect of the present invention provides an improved engineered transaminase polypeptide. This engineered polypeptide is developed by an artificial process of directed evolution with a certain number of mutations such as substitution, insertion or deletion of amino acid residues. In order to obtain a transaminase active for the reaction shown in FIG. 2, the inventors screened an engineered transaminase enzyme library developed by Enzymaster (Ningbo) Bioengineering Co. Ltd., and identified a transaminase variant with the sequence shown in SEQ ID NO: 2 which is active for the reaction shown in FIG. 2. SEQ ID NO: 2 is an engineered transaminase variant developed based on a wild-type transaminase from Aspergillus fumigatus. However, SEQ ID NO:2 shows low activity and stereoselectivity for substrate S1 and poor solvent tolerance. In the tests by the inventors, when using SEQ ID NO:2 for the reaction shown in Figure. 2, the yield was 35% after 24 hours at the reaction condition where substrate S1 loading was 5 g/L and enzyme loading was 10 g/L. And high concentrations of cosolvents such as methanol or DMSO had an inhibitory effect on SEQ ID NO: 2. If the activity of SEQ ID NO: 2 at 20% methanol was defined as 100%, the relative activities of SEQ ID NO: 2 at 35% methanol and 50% methanol were 59% and 43%, respectively; the relative activities at 20% DMSO, 35% DMSO and 50% DMSO were 74%, 23% and 8%, respectively. In the reaction system with methanol as the cosolvent, SEQ ID NO: 2 gave a dr value of 1.7 for the product, while it gave a dr value of 0.3 for the product when DMSO was used as cosolvent. In order to enable an industrial production of L1 using transaminase process, SEQ ID NO: 2 needs to be further engineered to enhance its activity, selectivity and stability.

The present invention utilizes computational biology techniques for model construction and virtual screening of the mutants of SEQ ID NO: 2. First, 112 stable mutants were obtained, after which 40 mutants potentially beneficial for enhancing the catalytic activity of the reaction shown in FIG. 2 were selected from the 112 stable mutants using the activity virtual screening technique. Next, the inventors subjected these 40 mutants predicted by the virtual screening to gene synthesis and recombinant expression in the laboratory, and experimentally verified their performance in catalyzing the reaction shown in FIG. 2 by setting appropriate reaction conditions. Among these 40 mutants, 15 were identified to have enhanced activity and/or selectivity, among which SEQ ID NO: 24 performed better. Compared to SEQ ID NO: 2, SEQ ID NO: 24 contains a mutation W183A. Based on these 15 experimentally validated mutants, the inventors conducted a new round of virtual screening of combinatorial libraries and identified seven beneficial mutations that could be suitable for combination. The inventors then constructed a combinatorial library recombining these seven beneficial mutations and screened the library using experimental methods to obtain the optimal mutant SEQ ID NO: 130 which contains the following amino acid substitutions compared to SEQ ID NO: 2: T52Y; Q53T; W183A; N190I.

The engineered transaminase polypeptide provided by the present invention comprises an amino acid sequence having activity to catalyze the reaction shown in FIG. 2 and having one or more residue differences compared to the SEQ ID NO: 2 at amino acid residue positions corresponding to the following: X52, X53, X115, X126, X146, X183, X190.

Further, compared to the SEQ ID NO: 2, the engineered transaminase polypeptide provided by the present invention comprises an amino acid sequence comprising at least one of the following features: T52Y, Q53TKFEH, N115GE, R126L, I146Q, W183AST, N190LI; or simultaneously, on the basis of these differences, 1, 2, 3, 4, 5, 6, 7 8, 9, 10, 11, 12, 13, 14, 15, 16, 18, 20, 21, 22, 23, 24, 25, or more insertions or deletions of amino acid residues.

More specifically, in some embodiments, an engineered transaminase polypeptide improved on the basis of SEQ ID NO:2 comprises a polypeptide of the group consisting of the amino acid sequences shown in SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58 60, 62, 64, 66, 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140 142, 144, 146, 148, 150, 152, 154, 156, 158.

In some embodiments, the improved engineered transaminase polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146, 148 150, 152, 154, 156, 158.

The identity between two amino acid sequences or two nucleotide sequences can be obtained by algorithms commonly used in the field, either by using the NCBI Blastp and Blastn software based on default parameters or by using the Clustal W algorithm (Nucleic Acid Research, 22(22):4673-4680, 1994). For example, using the Clustal W algorithm, the amino acid sequence identity between SEQ ID NO: 2 and SEQ ID NO: 130 is 98.7%.

In another aspect, the present invention provides polynucleotide sequences encoding engineered transaminase polypeptides. In some embodiments, the polynucleotide may be a portion of an expression vector having one or more control sequences for expression of the engineered transaminase polypeptide. In some embodiments, the polynucleotide may comprise polynucleotide sequence corresponding to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 7173, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143 145, 147, 149, 151, 153, 155, 157.

As known to those of skill in the art, due to the degeneracy of the nucleotide codons, the polynucleotide sequences encoding the amino acid sequences of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146 148, 150, 152, 154, 156, 158 are not limited to SEQ ID No:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131,133, 135, 137, 139, 141, 143, 145, 147, 149, 151, 153, 155, 157. The nucleic acid sequence encoding the engineered transaminase of the present invention may also be any other nucleic acid sequence encoding the amino acid sequence shown in the sequences of SEQ ID No: 2, 4, 6, 8, 10, 12, 14, 16, 18, 20, 22, 24, 26, 28, 30, 32, 32, 36, 38, 40, 34, 36, 38, 40, 42, 44, 46, 48, 50, 52, 54, 56, 58, 60, 62, 64, 66, 68, 70, 72, 74, 76, 78, 80, 82, 84, 86, 88, 100, 102, 104, 106, 108, 110, 112, 114, 116, 118, 120 122, 124, 126, 128, 130, 132, 134, 136, 138, 140, 142, 144, 146, 148, 150, 152, 154, 156, 158.

In another aspect, the present disclosure provides expression vectors and host cells comprising a polynucleotide encoding an engineered transaminase or capable of expressing an engineered transaminase. In some embodiments, the host cell may be a bacterial host cell, such as E. coli. The host cell can be used to express and isolate the engineered transaminases described herein, or optionally used directly to reactively transform the substrate into a product.

In some embodiments, engineered transaminases in the form of intact cells, crude extracts, isolated polypeptides, or purified polypeptides may be used alone, or in immobilized form (e.g., immobilized on a resin).

The engineered transaminase polypeptides disclosed herein catalyze the conversion of the ketone substrate shown in structural formula XI to the amine product shown in structural formula I.

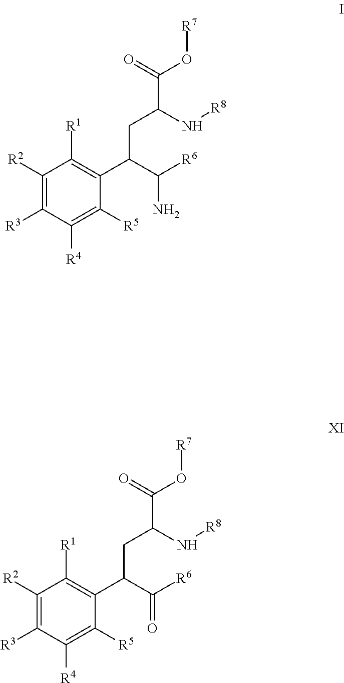

wherein the groups R¹, R², R³, R⁴, R⁵ can be optionally substituted —H, C₁-C₆ hydrocarbon group, halogen (e.g. —F, —Cl, —Br, —I), —NO₂, —NO—NO, —SO₂R′ or —SOR′, —SR′, —NR′R′, —OR′, —CO₂R′ or —COR′, —C(O)NR′—C(O)NR′, —SO₂NH₂ or —SONH₂, —CN, CF₃; R⁶ can be a C₁-C₆ hydrocarbon group, C₁-C₆ haloalkyl, C₁-C₆ hydroxy-substituted hydrocarbon; R⁷ can be C₁-C₆ hydrocarbon group, C₁-C₆ haloalkyl, C₁-C₆ hydroxy-substituted hydrocarbon; R¹ can be CBZ protecting group, BOC protecting group, Fomc protecting group, Bn protecting group, methyl(ethyl)oxycarbonyl protecting group. wherein each R′ is independently selected from —H or C₁-C₄ hydrocarbon group.

The amine product shown in structural formula I can be one of, or a mixture of the chiral amine products shown in structural formulae II-V.

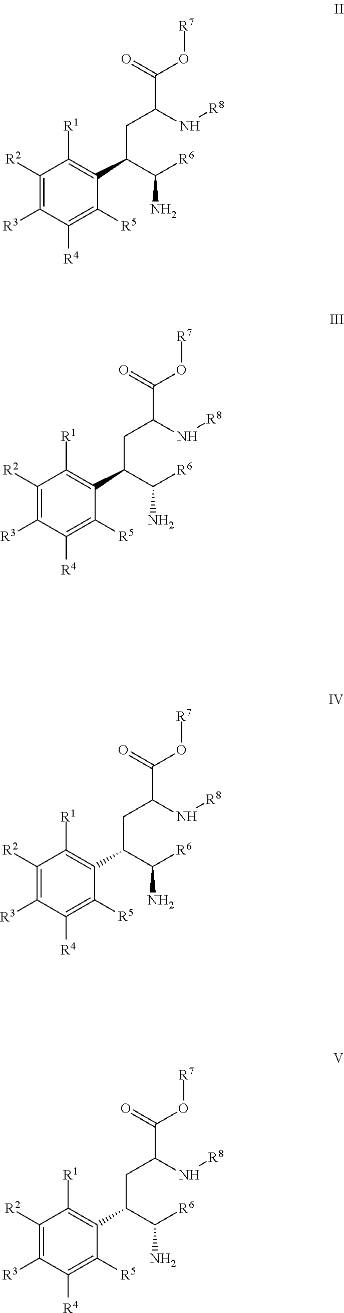

Depending on the activity of the ester group in the structure of compound I, under suitable reaction conditions, the amine product shown in structural formula I, generated by enzymatic catalysis, can spontaneously form a ring to produce a lactam shown in structural formula VI.

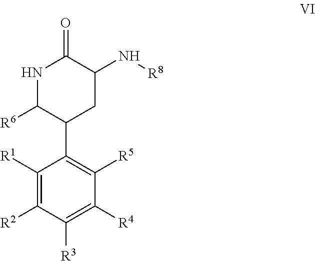

The chiral amine product shown in structural formula VI can be one of, or a mixture of the following chiral amine products shown in structural formulae VII-X.

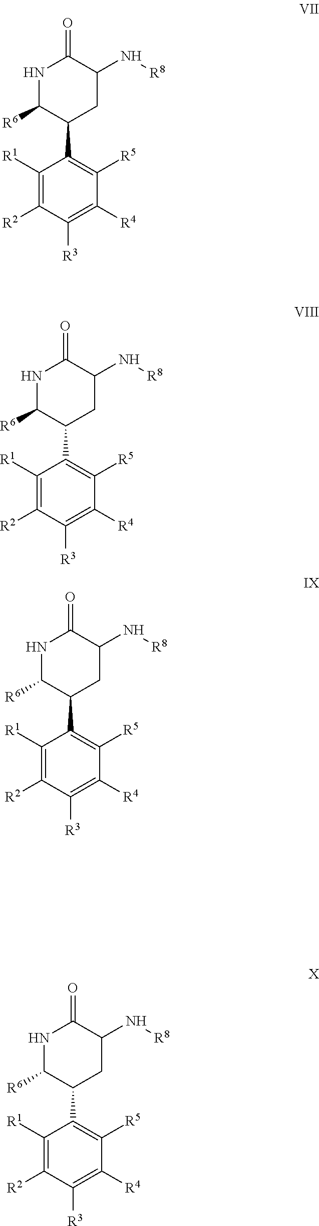

The corresponding substrate structural formula XI for the chiral amine products shown in structural formula I-X that can be catalyzed by the engineered transaminase polypeptide disclosed herein is:

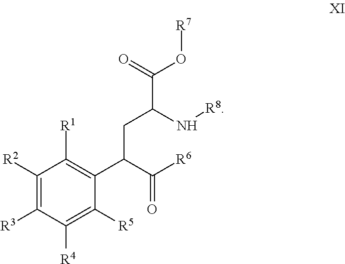

Preferably, the engineered transaminase polypeptide disclosed herein has significant activity to substrate S1, which has the following structural formula shown below:

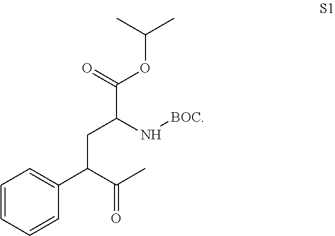 [SMILES: CC(=O)C(CC(NC(=O)OC(C)(C)C)C(=O)OC(C)C)c1ccccc1]

S1 may contain four different isomers ST1, ST2, SD1 or SD2 as follows.

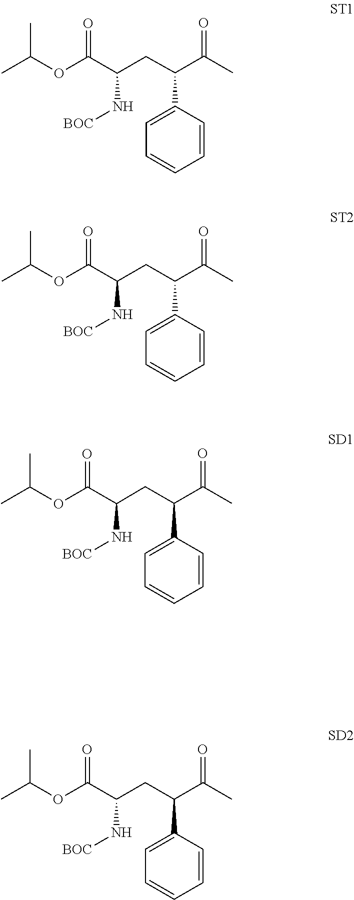

The engineered transaminase polypeptide disclosed in the present invention converts S1 to I1.

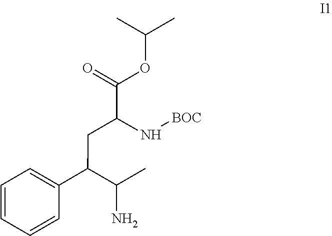 [SMILES: CC(C)OC(=O)C(CC(c1ccccc1)C(C)N)NC(=O)OC(C)(C)C]

I1 may contain the following four different isomers IT1, IT2, ID1 or ID2.

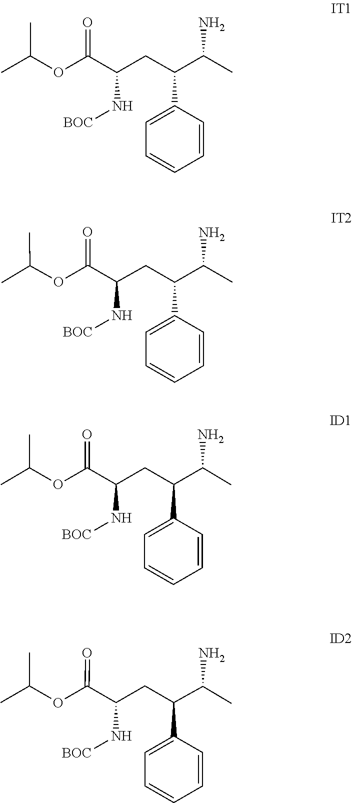

The compounds shown as structural formula IT represent IT1 and/or IT2:

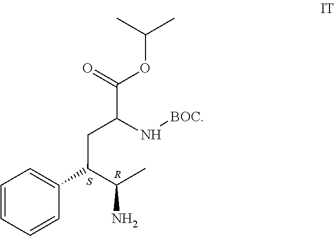

The ester bonds in the I1 structure can spontaneously break and a ring structure forms, resulting in the formation of the compounds shown as structural formula T1, T2, D1 or D2.

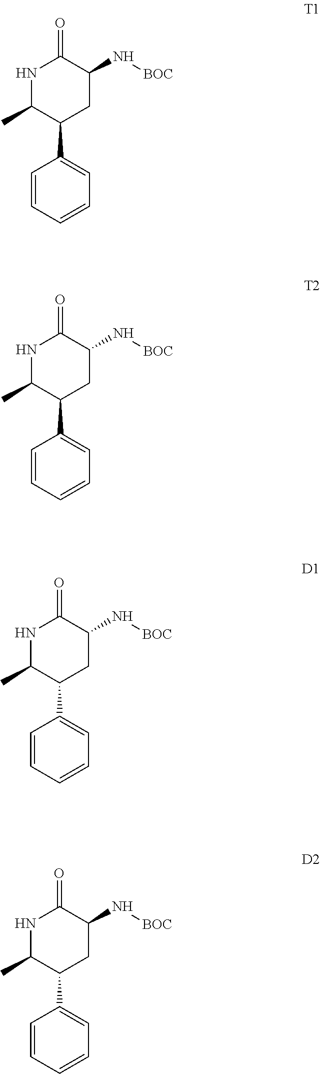 [SMILES: C[C@H]1NC(=O)[C@@H](NC(=O)OC(C)(C)C)C[C@H]1c1ccccc1 ; C[C@H]1NC(=O)[C@H](NC(=O)OC(C)(C)C)C[C@H]1c1ccccc1 ; C[C@H]1NC(=O)[C@H](NC(=O)OC(C)(C)C)C[C@@H]1c1ccccc1 ; C[C@H]1NC(=O)[C@@H](NC(=O)OC(C)(C)C)C[C@@H]1c1ccccc1]

Among the products generated from substrate S1 catalyzed by the engineered transaminase polypeptide disclosed in the present invention, the products in excess are IT1 and IT2 and subsequently T1 and T2. T1 and T2 are represented by the structural formula shown as L1.

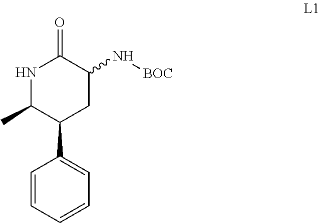 [SMILES: C[C@H]1NC(=O)C(NC(=O)OC(C)(C)C)C[C@H]1c1ccccc1]

In the present invention, the “diastereomeric ratio” (i.e., dr) is the ratio of the sum of the concentrations of the diastereomeric compound T1 and compound T2 to the sum of the concentrations of the diastereomeric compound D1 and compound D2 in the product, calculated by the formula: dr=[T1+T2]/[D1+D2].

In some embodiments, the engineered transaminase polypeptide has at least 80%, 81%, 82%, 83%, 84%, 85%, 86%, 87%, 88%, 89%, 90%, 91%, 92%, 93%, 94%, 95%, 96%, 97%, 98%, 99% or more sequence identity compared to SEQ ID NO:2 and is capable of converting compound S1 into one or more of the amine products of compounds T1, T2, D1, D2.

In some embodiments, the dr value of the product (i.e., [T1+T2]/[D1+D2]) is at least 1, 2, 3, 5, 10, 20, 30, 40, 50, 60, 70, 80, 90, 100, or higher.

Specific embodiments of the engineered transaminase polypeptide for use in the method are provided further in the detailed description. An improved engineered transaminase polypeptide available in the above methods may comprise amino acid sequence selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146 148, 150, 152, 154, 156, 158. Either of the methods of using an engineered polypeptide for preparing a compound of Formula I, Formula VI, Formula I1, or Formula L1 as disclosed herein may be performed under a range of suitable reaction conditions, said range of suitable reaction conditions including, but not limited to, a range of amino donors, pH, temperature, buffers, solvent systems, substrate loadings, peptide loadings, cofactor loadings, pressures, and reaction times. For example, in some embodiments, preparation of compounds of Formula T1 and/or T2 can be performed, wherein suitable reaction conditions include (a) a loading of about 10 g/L to 100 g/L of substrate S1, (b) a loading of about 1 g/L to 50 g/L of the engineered peptide, (c) a loading of about 0.1 M to 4.0 M of isopropylamine, (d) a pH of about 7.0 to 11.5, (e) a temperature of about 10° C. to 65° C. and (f) 0% to 70% solvent. Organic solvents described herein include, but are not limited to, methanol, dimethyl sulfoxide (DMSO), acetonitrile (ACN), dimethyl formamide (DMF), methyl tert-butyl ether (MTBE), isopropyl acetate, ethanol, propanol, isopropyl alcohol (IPA) or a mixture of two or more of them.

### DETAILED DESCRIPTION

### Definitions

With respect to this disclosure, unless otherwise expressly defined, the technical terms and scientific terms used in the specification herein have the meanings commonly understood by those of ordinary skill in the art.

The terms “protein,” “polypeptide,” and “peptide” are used interchangeably herein to refer to a polymer of at least two amino acids covalently linked by an amide bond, regardless of length or post-translational modifications (e.g., glycosylation, phosphorylation, lipidation, myristoylation, ubiquitination, etc.). The definition includes D-amino acids and L-amino acids, and mixtures of D-amino acids and L-amino acids.

“Engineered transaminase,” “engineered transaminase polypeptide,” “modified transaminase polypeptide,” “improved transaminase polypeptide,” and “engineered polypeptide” are used interchangeably herein.

“cells” or “wet cells” refer to a host cell that expresses a polypeptide or engineered polypeptide, including a wet cell obtained by the preparation process shown in Example 2.

The terms “polynucleotide” and “nucleic acid” are used interchangeably herein.

As used herein, “cofactor” refers to a non-protein compound that acts in combination with an enzyme in a catalytic reaction. “cofactors” are intended to include pyridoxal phosphate (pyridoxal-5′-phosphate, or PLP), pyridoxine (pyridoxol, PN), pyridoxal (PL), pyridoxamine (PM), pyridoxine phosphate (PNP) and pyridoxamine phosphate (PMP) of the vitamin B6 family of compounds, which are sometimes referred to as coenzymes. “PLP”, “pyridoxal phosphate”, “pyridoxal 5′-phosphate”, “PYP” and “P5P” are used interchangeably herein to refer to compounds that are used as cofactors in enzyme-catalyzed reactions.

“Coding sequence” refers to the nucleic acid portion (e.g., a gene) that encodes an amino acid sequence of a protein.

“Naturally occurring” or “wild-type” refers to the form found in nature. For example, a naturally occurring or wild-type polypeptide or polynucleotide sequence is a sequence that exists in an organism that is isolable from a natural source and has not been intentionally modified by artificial manipulation.

“Recombinant” or “engineered” or “non-naturally occurring”, when used to refer to, for example, a cell, nucleic acid or polypeptide, refers to a material that is, or corresponds to, the natural or inherent form of the material, that has been altered in a manner not found in nature, or is identical to it but is produced or obtained from synthetic material and/or by manipulation using recombinant technology.

“Sequence identity” is used herein to refer to a comparison between polynucleotides or polypeptides (“sequence identity” is usually expressed as a percentage) and is determined by comparing two optimally aligned sequences on a comparison window, where the portion of the polynucleotide or polypeptide sequence in the comparison window may include additions or deletions (i.e., gaps) compared to the reference sequence for optimal alignment of the two sequences. The percentage may be calculated by determining the number of positions where identical nucleic acid bases or amino acid residues occur in the two sequences to produce the number of matching positions, dividing the number of matching positions by the total number of positions in the comparison window and multiplying the result by 100 to obtain the percentage of sequence identity. Optionally, the percentage may be calculated by determining the number of positions where the same nucleic acid base or amino acid residue is present in both sequences or the number of positions where the nucleic acid base or amino acid residue is aligned with gaps to obtain the number of matching positions, dividing that number of matching positions by the total number of positions in the comparison window, and multiplying the result by 100 to obtain the percentage of sequence identity. Those of skill in the art will recognize that many established algorithms exist that can be used to align two sequences. The optimal alignment of sequences for comparison can be done, for example, by the local homology algorithm of Smith and Waterman, 1981, Adv. Appl. Math. 2:482, by the homology comparison algorithm of Needleman and Wunsch, 1970, J. Mol. Biol. 48:44, by the homology comparison algorithm of Pearson Lipman, 1988, Proc. Natl. Acad. Sci. USA 85:2444, by computerized implementations of these algorithms (GAP, BESTFIT, FASTA or TFASTA in the GCGWisconsin package) or by visual inspection (see, generally, Current Protocols in Molecular Biology, edited by F. M. Ausubel et al, Current Protocols, a joint venture between Greene Publishing Associates Inc. and John Wiley & Sons, Inc. (1995 supplement) (Ausubel)). Examples of algorithms suitable for determining sequence identity and percent sequence similarity are the BLAST and BLAST2.0 algorithms, which are described in Altschul et al, 1990, J. Mol. Biol. 215:403-410 and Altschul et al, 1977, Nucleic Acids Res. 3389-3402, respectively. The software used to perform the BLAST analysis is publicly available through the National Center for Biotechnology Information (NCBI) website. The algorithm involves first identifying high scoring sequence pairs (HSP) by identifying short words of length W in the query sequence, which either match or satisfy some positive-valued threshold score T when aligned with a word of the same length in the database sequence. T is referred to as, the neighborhood word score threshold (Altschul et al., as described above). These initial neighborhood word hits serve as seeds for initiating searches to find longer HSPs that contain them. The word hits are then extended in both directions along each sequence for as far as the cumulative alignment score can be increased. For nucleotide sequences, the cumulative scores are calculated using the parameters M (reward score for matched pair of residues; always>0) and N (penalty score for mismatched residues; always <0). For amino acid sequences, a scoring matrix is used to calculate the cumulative score. The extension of the word hits in each direction are halted when: the cumulative alignment score falls off by the quality X from its maximum achieved value; the cumulative score goes 0 or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, the expected value (E) of 10, M=5, N=−4, and a comparison of both strands as a default value. For amino acid sequences, the BLASTP program uses as defaults the wordlength (W) of 3, the expected value (E) of 10 and the BLOSUM62 scoring matrix (see Henikoff and Henikoff, 1989, Proc Natl Acad Sci USA 89: 10915). Exemplary determination of sequence alignments and % sequence identity can employ the BESTFIT or GAP programs in the GCG Wisconsin Software package (Accelrys, Madison WI), using the default parameters provided.

“Reference sequence” refers to a defined sequence that is used as a basis for sequence comparison. The reference sequence may be a subset of a larger sequence, for example, a full-length gene or a fragment of a polypeptide sequence. In general, a reference sequence is at least 20 nucleotides or amino acid residues in length, at least 25 residues long, at least 50 residues in length, or the full length of the nucleic acid or polypeptide. Because two polynucleotides or polypeptides may each (1) comprise a sequence (i.e., a portion of the complete sequence) that is similar between two sequences, and (2) may further comprise sequences that is divergent between the two sequences, sequence comparisons between two (or more) polynucleotides or polypeptides are typically performed by comparing the sequences of the two polynucleotides or polypeptides over a “comparison window” to identify and compare local regions of sequence similarity. In some embodiments, a “reference sequence” is not intended to be limited to a wild-type sequence, and may comprise engineered or altered sequences. “Comparison window” refers to a conceptual segment of at least about 20 contiguous nucleotide positions or amino acid residues, wherein the sequence may be compared to a reference sequence of at least 20 contiguous nucleotides or amino acids and wherein the portions of the sequence in the comparison window may comprise 20% or less additions or deletions (i.e., gaps) compared to the reference sequence (which does not comprise additions or deletions) for optimal alignment of the two sequences. The comparison window can be longer than 20 contiguous residues, and optionally include 30, 40, 50, 100 or more residues.

In the context of the numbering for a given amino acid or polynucleotide sequence, “corresponding to,” “reference to” or “relative to” refers to the numbering of the residues of a specified reference when the given amino acid or polynucleotide sequence is compared to the reference sequence. In other words, the residue number or residue position of a given sequence is designated with respect to the reference sequence, rather than by the actual numerical position of the residue within the given amino acid or polynucleotide sequence. For example, a given amino acid sequence such as the amino acid sequence of an engineered transaminase can be aligned to a reference sequence, by introducing gaps to optimize the residue match between the two sequences. In these cases, the numbering of the residue in a given amino acid or polynucleotide sequence is made with respect to the reference sequence to which it has been aligned, despite the presence of a gap position.

An “amino acid difference” or “residue difference” refers to a difference in an amino acid residue at a position of a polypeptide sequence relative to an amino acid residue at a corresponding position in a reference sequence. The position of an amino acid difference is generally referred to herein as “Xn”, where n refers to the corresponding position in the reference sequence on which the residue difference is based. For example, “residue difference at position X183 compared to SEQ ID NO:2” refers to the difference in amino acid residues at the polypeptide position corresponding to position 183 of SEQ ID NO:2. Thus, if the reference polypeptide of SEQID NO:2 has a tryptophan at position 183, then “residue difference at position X183 compared to SEQ ID NO:2” refers to an amino acid substitution at any residue other than a tryptophan at the position of the polypeptide corresponding to position 183 of SEQ ID NO:2. In most of the examples herein, the specific amino acid residue difference at the position is indicated as “XnY”, wherein “Xn” refers to the corresponding position as described above, and “Y” is the single letter identifier of the amino acid found in the engineered polypeptide (i.e., a different residue than in the reference polypeptide). In some examples (e.g., in Table 2), the present disclosure also provides specific amino acid differences indicated by the conventional symbol “AnB”, where A is a single letter identifier of a residue in the reference sequence, “n” is the number of residue position in the reference sequence, and B is the single letter identifier for the residue substitution in the sequence of the engineered polypeptide. In some examples, the polypeptide of the present disclosure may comprise one or more amino acid residue differences relative to a reference sequence, which is indicated by a list of specific positions at which residue differences are present exist relative to the reference sequence.

“Deletion” refers to the modification of a polypeptide by removing one or more amino acids from a reference polypeptide. Deletions can include the removal of one or more amino acids, two or more amino acids, five or more amino acids, ten or more amino acids, fifteen or more amino acids, or twenty or more amino acids, up to 10% of the total number of amino acids of the enzyme, or up to 20% of the total number of amino acids making up the reference enzyme while retaining the enzymatic activity of the engineered transaminase polypeptide for the reaction shown in FIG. 2. Deletion may involve the internal portion and/or the terminal portion of the polypeptide. In various embodiments, deletions may include a contiguous segment or may be discontinuous.

“Insertion” refers to a modification of the polypeptide by adding one or more amino acids from the reference polypeptide. In some embodiments, the engineered polypeptides disclosed herein include one or more amino acid insertions into naturally occurring transaminase polypeptides, as well as insertions of one or more amino acids to other engineered polypeptides. The insertion may be made in the internal portion of the polypeptide, or into the carboxyl or amino terminus. As used herein, insertions include fusion proteins known in the art. The insertion may be a contiguous segment of amino acids or be separated by one or more amino acids in naturally-occurring or engineered polypeptides.

As used herein, “fragment” refers to a polypeptide having an amino terminal and/or carboxy terminal deletion, but where the remaining amino acid sequence is identical to the corresponding position in the sequence. Fragments may be at least 10 amino acids long, at least 20 amino acids long, at least 50 amino acids long or longer, and up to 70%, 80%, 90%, 95%, 98%, and 99% of the full-length engineered polypeptide.

An “isolated polypeptide” or “purified polypeptide” refers to a polypeptide that is substantially separated from other substances with which it is naturally associated, such as proteins, lipids, and polynucleotides. The term comprises polypeptides that have been removed or purified from their naturally occurring environment or expression system (e.g., in host cells or in vitro synthesis). Engineered transaminase polypeptides may be present in the cell, in the cell culture medium, or prepared in various forms, such as lysates or isolated preparations. As such, in some embodiments, the engineered transaminase polypeptide may be an isolated polypeptide.

“Chiral center” refers to a carbon atom connecting four different groups.

“Stereoselectivity” refers to the preferential formation of one stereoisomer over the other in a chemical or enzymatic reaction. Stereoselectivity can be partial, with the formation of one stereoisomer is favored over the other; or it may be complete where only one stereoisomer is formed. When stereoisomers are diastereomers, the stereoselectivity is referred to as diastereomer selectivity or diastereoselectivity, and the ratio of one (group of) diastereomer(s) relative to another (group of) diastereomer(s) is typically reported as the “diastereomeric ratio” (dr). This ratio (dr) is optionally derived therefrom according to the following formula: {concentration of major diastereomers}/{concentration of minor diastereomers}.

The terms “stereoisomers”, “stereoisomeric forms” and similar expressions are used interchangeably herein to refer to all isomers resulting from a difference in orientation of atoms in their space only. These include enantiomers and isomers of compounds with more than one chiral centers that are not mirror images of one another (i.e., “diastereomers”).

“Improved enzymatic properties” refers to an improved polypeptide showing any enzymatic properties compared to a reference sequence that evolves the starting transaminase SEQ ID No: 2. Desired improved enzyme properties include, but are not limited to, enzyme activity (which may be expressed as a percentage of product production), thermal stability, solvent stability (e.g., stability against alcohols), pH activity characteristics, cofactor requirements, tolerance to inhibitors (e.g., substrate or product inhibition), stereospecificity, and stereoselectivity.

“Reaction yield” refers to the molar percentage of product produced in the reaction system as a percentage of the starting substrate (charged at the beginning of the reaction) within a period of time under specified reaction conditions. Thus, “enzymatic activity” or “activity” of a transaminase or engineered polypeptide can be expressed as the “reaction yield”. The reaction yield is generally calculated by sampling to measure the molar concentration of the product and the molar concentration of the starting substrate in the reaction system: {molar concentration of product}/{molar concentration of starting substrate}.

“Thermostable” means that the engineered polypeptide maintains similar activity after exposure to elevated temperatures (e.g., 65° C. or higher) for a sustained period of time (e.g., 0.5 h or longer) compared to the starting polypeptide template.

“Solvent stable” or “solvent tolerant” means that the engineered polypeptide maintains similar activity after exposure to different concentrations (e.g., 5-99%) of solvents (methanol, ethanol, isopropanol, dimethyl sulfoxide (DMSO), tetrahydrofuran, 2-methyl tetrahydrofuran, acetone, toluene, butyl acetate, methyl tert-butyl ether, etc.) for a period of time (e.g., 0.5-24 h) compared to the starting polypeptide template.

“Suitable reaction conditions” refers to those conditions (e.g., range of enzyme loading, substrate loading, amino donor loading, cofactor loading, temperature, pH, buffer, cosolvent, etc.) in the biocatalytic reaction system, under which the engineered polypeptide of the present disclosure converts the substrate to the desired product compound. Exemplary “suitable reaction conditions” are provided in the present disclosure and exemplified by embodiments. Compounds may be identified by their chemical structure and/or chemical name. When the chemical structure and chemical name conflict, the chemical structure determines the identity of the compound.

### Directed Evolutionary Process and the Resulting Engineered Transaminases Developed

The engineered transaminase polypeptide disclosed in the present invention was developed by a creative directed evolution process with a certain number of amino acid residue substitutions, insertions or deletions. The transaminase corresponding to SEQ ID NO:2 was tested by the inventors and it was active against S1, with low activity, poor diastereomer selectivity and poor solvent tolerance. In order to develop an engineered transaminase with excellent performance suitable for the reaction shown in FIG. 2, an directed evolution process with 3 stages were executed, as shown in Table 1. The focus of each stage of development was different and different screening assay conditions were applied; the optimal engineered transaminase peptides obtained in each stage are shown in Table 2.

TABLE 1
The 3 stages of directed evolution

| Directed | | |
|---|---|---|
| evolution | | Assay conditions for screening enzyme |
| stage | R&D focus | variants |
| I | Identification of enzyme variants | Enzyme solution 55% (v/v), substrate 5 g/L, |
| | active against S1 by screening an | DMSO 20% (v/v), isopropylamine 1M, PLP |
| | extant library of engineered | 0.5 g/L, sodium tetraborate 0.025M, pH 10.5, |
| | transaminases | 45° C. |
| II | Investigation of amino acid sites | Enzyme solution 30% (v/v), substrate 5 g/L, |
| | that have significant effects on | methanol 35% (v/v), isopropylamine 1M, PLP |
| | enzyme activity, stability and | 0.5 g/L, sodium tetraborate 0.025M, pH 10.5, |
| | selectivity with the aid of virtual | 45° C. |
| | screening technique | |
| III | Enhancement of activity, | Enzyme solution 30% (v/v), substrate 30 g/L, |
| | stability and selectivity | DMSO 35% (v/v), acetonitrile 15% (v/v), |
| | | isopropylamine 1M, PLP 0.5 g/L, sodium |
| | | tetraborate 0.025M, pH 10.5, 55° C. |

The main objective of stage I was to screen an extant library of engineered transaminase enzymes that had been developed to find a transaminase catalyst that was active in catalyzing the generation of the product L1 from substrate S1 for direct industrial application or serving as a starting variant for further development through directed evolution. SEQ ID NO:2 was identified by the inventors as the most suitable starting variant, which was developed from a wild-type transaminase derived from Aspergillus fumigatus (NCBI: XP_748821.1). Table 2 lists the residue differences of SEQ ID NO:2 compared to the wild type enzyme, and the sequence identity compared to the wild type enzyme. The amino acid sequence identity was calculated using the Clustal W algorithm (Nucleic Acid Research, 22(22):4673-4680, 1994). SEQ ID NO:2 was modified by directed evolutionary techniques to further increase the activity, stability, selectivity and other properties for industrial applications.

TABLE 2
Differences between SEQ ID NO: 2 and wild-type enzymes

| | Amino | | Amino acid sequence |
|---|---|---|---|
| | acid | | identity relative to |
| Polynucleotides | SEQ ID | Amino acid residue differences relative to wild- | wild-type enzyme |
| SEQ ID NO | NO | type enzyme (NCBI: XP_748821.1) | (NCBI: XP_748821.1) |
| 1 | 2 | L36F; E49P; M52T; H53Q; D55W; L56A; D59T; | 87.9% |
| | | V60T; Q76K; I78L; M85S; K97R; N98K; F113L; | |
| | | E115N; V116I; S128R; K129Q; Y134P; N136P; | |
| | | V148L; N153K; L155R; E159H; L181F; T187R; | |
| | | L190N; M194A; T199E; N209H; R235E; K240Q; | |
| | | I253T; Q262A; C272T; T273S; D291G; P312N; | |
| | | G323N | |

The main objective of stage 1 was to find amino acid mutations that have significant effects on enzyme activity, stability and selectivity, and to provide data support for subsequent library design for directed evolution. The present invention performed a virtual screening of mutants of SEQ ID NO:2 by utilizing bioinformatics and computational biology techniques, and the general flow of this virtual screening method is shown below.

The process of virtual screening is as follows and consists of steps shown in FIG. 3.

Step 1 Homology modeling: SEQ ID NO:2 was modeled with PDBID 4UUG as the template by Yasara software to generate a 3D modeling, and the modeling parameters are shown in Table 3.

TABLE 3
Homology modeling parameters

| Modeling speed (slow = best): | Slow |
|---|---|
| Number of PSI-BLAST iterations in template | 3 |
| search (PsiBLASTs): | |
| Maximum allowed (PSI-)BLAST E-value to consider | 0.5 |
| template (EValue Max): | |
| Maximum number of templates to be used | 5 |
| (Templates Total): | |
| Maximum number of templates with same sequence | 1 |
| (Templates SameSeq): 1 | |
| Maximum oligomerization state (OligoState): | 4 (tetrameric) |
| Maximum number of alignment variations per | 5 |
| template: (Alignments): | |
| Maximum number of conformations tried per | 50 |
| loop (LoopSamples): | |
| Maximum number of residues added to the termini | 10 |
| (TermExtension): | |

Step 2 Docking via Autodock: The four substrate isomers ST1, ST2, SD1, SD2 were docked with the target enzyme by the autodock method in Yasara software to obtain the enzyme-substrate complex (FIG. 4), and amino acids within 5 Å from the substrate were selected as candidate mutagenesis sites (T52, Q53, T60, L113, N115, R126, L141, L143, 1146, L148, W183, N190, G215, S273, T274, A275).

Step 3 Virtual screening for stability via Rosetta software: Each candidate mutagenesis site selected in the previous step was subject to saturation mutagenesis, and virtual screening of each mutant for stability was performed by Cartesian_ddg algorithm in Rosetta software. The number of mutants for virtual screening was 16*19=304 single-site mutations. The virtual screening yielded 112 single-site mutations that were favorable for stability, and the results are shown in Table 4.

TABLE 4
Single-site mutations predicted by virtual
screening to be beneficial for stability

| | Amino acid residues |
|---|---|
| Site | for substitution |
| T52 | FYASLIED |
| Q53 | TKFEHGLNDE |
| T60 | SAILDEFY |
| L113 | ISVMWFYA |
| N115 | GEWQRD |
| R126 | CYHLIKED |
| L141 | EIVYAST |
| L143 | SVHIAKH |
| I146 | NLQDEFA |
| L148 | IASTKHR |
| W183 | ASLTFYIDERK |
| N190 | LITQED |
| G215 | ASTIL |
| S273 | ATLI |
| T274 | ASLI |
| A275 | SLITRK |

The stability of these mutations was judged by the following criteria: ΔΔG≤−1 kcal/mol for stable mutants; ΔΔG≥1 kcal/mol for unstable mutants; −1 kcal/mol<ΔΔG<1 kcal/mol for void mutants. This criterion is also applicable to the judging of stability results derived from other calculation methods.

Step 4 Virtual screening for activity: The reaction energy barrier is the minimum energy required to reach the activated molecule from the reactant molecule, and the size of the energy barrier can indicate the difficulty of reaction occurrence. Therefore, the present invention constructed a process based on empirical valence bonding theory to realize the bulk calculation of reaction energy barriers, and the mutations with enhanced activity were obtained by comparing the difference in calculated reaction energy barriers between SEQ ID NO:2 and the mutants. A total of 40 mutations with enhanced activity for the target product T1 or T2 were obtained in this virtual screening step, and the results are shown in Table 5.

TABLE 5
Single-site mutations predicted to be beneficial
for catalytic activity towards T1 or T2

| | Amino acid residues |
|---|---|
| Site | for substitution |
| T52 | FY |
| Q53 | TKFEHGL |
| L113 | ISVMW |
| N115 | GEW |
| R126 | CYHLI |
| L141 | GEIVY |
| L143 | SVH |
| I146 | NLQ |
| W183 | ASLT |
| N190 | LIT |

The final results of the experimental screening showed that 15 of the 40 virtually predicted mutations showed elevated dr values with similar or significantly higher activity for the generation of T1 or T2 compared to SEQ ID NO: 2. Among them, a superior mutant enzyme is SEQ ID NO: 24 with mutation W183A, examples of which are shown in Example 5. The 15 beneficial mutations and their corresponding activity and selectivity are given in Table 6.

TABLE 6
Activity and selectivity of experimentally validated single-site
mutations beneficial for catalytic production of T1 or T2

| | Mutation | Fold of activity | | |
|---|---|---|---|---|
| | compared to | enhancement | | |
| | SEQ ID NO: | Compared to | Product | |
| SEQ ID NO | 2 | SEQ ID NO: 2 | dr value | Assay conditions |
| 4 | T52Y | 0.81 | 7.9 | Assay condition |
| | | | | for stage II |
| | | | | in Table 1 |
| 6 | Q53T | 0.9 | 5.3 | Assay condition |
| | | | | for stage II |
| | | | | in Table 1 |
| 8 | Q53K | 1.1 | 2.4 | Assay condition |
| | | | | for stage II |
| | | | | in Table 1 |
| 10 | Q53F | 0.7 | 3.2 | Assay condition |
| | | | | for stage II |
| | | | | in Table 1 |
| 12 | Q53E | 0.8 | 2.4 | Assay condition |
| | | | | for stage II |
| | | | | in Table 1 |
| 14 | Q53H | 1.2 | 3.5 | Assay condition |
| | | | | for stage II |
| | | | | in Table 1 |
| 16 | N115G | 1.2 | 3.1 | Assay condition |
| | | | | for stage II |
| | | | | in Table 1 |
| 18 | N115E | 0.8 | 3.3 | Assay condition |
| | | | | for stage II |
| | | | | in Table 1 |
| 20 | R126L | 1.3 | 3.4 | Assay condition |
| | | | | for stage II |
| | | | | in Table 1 |
| 22 | I146Q | 1.7 | 2.1 | Assay condition |
| | | | | for stage II |
| | | | | in Table 1 |
| 24 | W183A | 6.1 | 7.4 | Assay condition |
| | | | | for stage II |
| | | | | in Table 1 |
| 26 | W183S | 7.2 | 4.6 | Assay condition |
| | | | | for stage II |
| | | | | in Table 1 |
| 28 | W183T | 4.8 | 3.5 | Assay condition |
| | | | | for stage II |
| | | | | in Table 1 |
| 30 | N190L | 0.8 | 2.0 | Assay condition |
| | | | | for stage II |
| | | | | in Table 1 |
| 32 | N190I | 3.8 | 2.9 | Assay condition |
| | | | | for stage II |
| | | | | in Table 1 |

Mutants or Mutagenesis libraries can be constructed using either Site-specific mutagenesis PCR or multi-site mutagenesis PCR as is common in the field (see “Mutagenesis and Synthesis of Novel Recombinant Genes Using PCR”, Chapter 32, in PCR Primer, 2nd edition (eds. Dieffenbach and Dveksler). ColdSpring Harbor Laboratory Press, Cold Spring Harbor, NY, USA, 2003.)

The main objective of stage III was to obtain an enzyme with significantly higher activity, selectivity and solvent tolerance (stability). In the previous two stages, the inventors found that there were advantages and disadvantages to use either methanol or DMSO as a single cosolvent for substrate S1 in the reaction. If methanol is used as a single cosolvent, the substrate has a better solubility but it is more easily to undergo hydrolysis; if DMSO is used as a single cosolvent, the solubility of the substrate S1 in the reaction system is lower, which affects the in situ epimerization of the substrate isomers (i.e., SD1 and SD2 are converted to ST1 and ST2). In order to solve the defects caused by the use of a single cosolvent in the reaction, the inventors creatively used a mixture of DMSO/ACN instead of a single cosolvent in the reaction, since the solubility of substrate S1 in ACN is much higher than that of DMSO, and the substrate is not easily hydrolyzed in both DMSO and ACN systems. The advantage of using a mixture of cosolvents is that the solubility of substrate S1 in the reaction system can be increased to promote the in situ epimerization of the substrate, and the hydrolysis of the substrate caused by the use of alcohol solvents can be greatly reduced, and the inhibition of enzyme activity by high concentration of acetonitrile when used as a single cosolvent can be avoided to a certain extent. In order to engineer the enzymes with improved performance, the 15 beneficial mutations obtained in stage II were combined in the library design to obtain a combinatorial mutagenesis library containing 1728 variant sequences which was subject to Rosetta virtual screening for stability and activity. The mutation combinations of variants ranking in the top 20% in terms of activity scoring were analyzed to obtain the probability of occurrence of dominant amino acid residues. The most suitable amino acid residues for each site are shown in Table 7.

TABLE 7
Predicted beneficial mutations suitable
for combination by virtual screening

| | Amino acid residues |
|---|---|
| Site | for substitution |
| T52 | Y |
| Q53 | T |
| R126 | L |
| I146 | Q |
| A183 | S |
| N190 | I |

Finally, the optimal mutations obtained for each site were combined into a combinatorial mutagenesis library which was constructed on the basis of SEQ ID NO:24 and screened using the stage III screening reaction conditions in Table 1, as exemplified in Example 7. SEQ ID NO: 130 was obtained with significantly enhanced activity and stereoselectivity. Compared to SEQ ID NO: 2, SEQ ID NO: 130 has 4 mutations: T52Y, Q53T, W113A and N190I. Table 8 shows the engineered transaminase polypeptides for each combination, its activity enhancement compared to SEQ ID NO: 2, and the corresponding dr values of the catalytically generated products.

TABLE 8
Activity of the engineered transaminase polypeptides corresponding to each
combinatorial mutation and the dr values of their catalyzed products

| | | Activity | | |
|---|---|---|---|---|
| SEQ | | enhancement | Product | |
| ID | Amino acid residue differences | compared to | dr | |
| NO | compared to SEQ ID NO: 2 | SEQ ID NO: 2 | value | Assay conditions |
| 34 | W183A; N190I; | 13.8 | 5.7 | Assay conditions for stage III in Table 1 |
| 36 | W183S; | 7.5 | 5.6 | Assay conditions for stage III in Table 1 |
| 38 | W183S; N190I; | 16.4 | 3.3 | Assay conditions for stage III in Table 1 |
| 40 | I146Q; W183A; | 6.1 | 5.7 | Assay conditions for stage III in Table 1 |
| 42 | I146Q; W183A; N190I; | 10.9 | 3.8 | Assay conditions for stage III in Table 1 |
| 44 | I146Q; W183S; | 8.7 | 3.9 | Assay conditions for stage III in Table 1 |
| 46 | I146Q; W183S; N190I; | 14.2 | 2.4 | Assay conditions for stage III in Table 1 |
| 48 | R126L; W183A; | 7.1 | 2.7 | Assay conditions for stage III in Table 1 |
| 50 | R126L; W183A; N190I; | 12.9 | 15.5 | Assay conditions for stage III in Table 1 |
| 52 | R126L; W183S; | 8.7 | 1.7 | Assay conditions for stage III in Table 1 |
| 54 | R126L; W183S; N190I; | 18.6 | 9.1 | Assay conditions for stage III in Table 1 |
| 56 | R126L; I146Q; W183A; | 5.7 | 2.4 | Assay conditions for stage III in Table 1 |
| 58 | R126L; I146Q; W183A; N190I; | 10.8 | 12.2 | Assay conditions for stage III in Table 1 |
| 60 | R126L; I146Q; W183S; | 8.1 | 1.3 | Assay conditions for stage III in Table 1 |
| 62 | R126L; I146Q; W183S; N190I; | 16.2 | 7.2 | Assay conditions for stage III in Table 1 |
| 64 | Q53T; W183A; | 7.2 | 3.6 | Assay conditions for stage III in Table 1 |
| 66 | Q53T; W183A; N190I; | 14.5 | 15.2 | Assay conditions for stage III in Table 1 |
| 68 | Q53T; W183S; | 10.6 | 2.8 | Assay conditions for stage III in Table 1 |
| 70 | Q53T; W183S; N190I; | 19.8 | 9.5 | Assay conditions for stage III in Table 1 |
| 72 | Q53T; I146Q; W183A; | 6.3 | 2.9 | Assay conditions for stage III in Table 1 |
| 74 | Q53T; I146Q; W183A; N190I; | 9.4 | 12.0 | Assay conditions for stage III in Table 1 |
| 76 | Q53T; I146Q; W183S; | 8.6 | 1.4 | Assay conditions for stage III in Table 1 |
| 78 | Q53T; I146Q; W183S; N190I; | 13.4 | 7.2 | Assay conditions for stage III in Table 1 |
| 80 | Q53T; R126L; W183A; | 6.2 | 9.0 | Assay conditions for stage III in Table 1 |
| 82 | Q53T; R126L; W183A; N190I; | 15.1 | 44.4 | Assay conditions for stage III in Table 1 |
| 84 | Q53T; R126L; W183S; | 9.3 | 1.1 | Assay conditions for stage III in Table 1 |
| 86 | Q53T; R126L; W183S; N190I; | 19.8 | 5.4 | Assay conditions for stage III in Table 1 |
| 88 | Q53T; R126L; I146Q; W183A; | 5.0 | 7.2 | Assay conditions for stage III in Table 1 |
| 90 | Q53T; R126L; I146Q; W183A; N190I; | 5.6 | 37.2 | Assay conditions for stage III in Table 1 |
| 92 | Q53T; R126L; I146Q; W183S; | 8.6 | 4.3 | Assay conditions for stage III in Table 1 |
| 94 | Q53T; R126L; I146Q; W183S; N190I; | 16.1 | 21.6 | Assay conditions for stage III in Table 1 |
| 96 | T52Y; W183A; | 11.5 | 7.0 | Assay conditions for stage III in Table 1 |
| 98 | T52Y; W183A; N190I; | 21.1 | 35.0 | Assay conditions for stage III in Table 1 |
| 100 | T52Y; W183S; | 19.3 | 4.2 | Assay conditions for stage III in Table 1 |
| 102 | T52Y; W183S; N190I; | 32.2 | 21.0 | Assay conditions for stage III in Table 1 |
| 104 | T52Y; I146Q; W183A; | 8.7 | 5.6 | Assay conditions for stage III in Table 1 |
| 106 | T52Y; I146Q; W183A; N190I; | 17.7 | 28.0 | Assay conditions for stage III in Table 1 |
| 108 | T52Y; I146Q; W183S; | 13.4 | 3.5 | Assay conditions for stage III in Table 1 |
| 110 | T52Y; I146Q; W183S; N190I; | 24.6 | 16.9 | Assay conditions for stage III in Table 1 |
| 112 | T52Y; R126L; W183A; | 9.6 | 21.1 | Assay conditions for stage III in Table 1 |
| 114 | T52Y; R126L; W183A; N190I; | 16.3 | >100 | Assay conditions for stage III in Table 1 |
| 116 | T52Y; R126L; W183S; | 13.9 | 8.2 | Assay conditions for stage III in Table 1 |
| 118 | T52Y; R126L; W183S; N190I; | 25.7 | 69.0 | Assay conditions for stage III in Table 1 |
| 120 | T52Y; R126L; I146Q; W183A; | 7.7 | 16.8 | Assay conditions for stage III in Table 1 |
| 122 | T52Y; R126L; I146Q; W183A; N190I; | 16.9 | 91.7 | Assay conditions for stage III in Table 1 |
| 124 | T52Y; R126L; I146Q; W183S; | 11.6 | 11.1 | Assay conditions for stage III in Table 1 |
| 126 | T52Y; R126L; I146Q; W183S; N190I; | 21.5 | 49.4 | Assay conditions for stage III in Table 1 |
| 128 | T52Y; Q53T; W183A; | 12.3 | 21.1 | Assay conditions for stage III in Table 1 |
| 130 | T52Y; Q53T; W183A; N190I; | 21.7 | >100 | Assay conditions for stage III in Table 1 |
| 132 | T52Y; Q53T; W183S; | 16.4 | 12.9 | Assay conditions for stage III in Table 1 |
| 134 | T52Y; Q53T; W183S; N190I; | 21.2 | 43.4 | Assay conditions for stage III in Table 1 |
| 136 | T52Y; Q53T; I146Q; W183A; | 8.8 | 16.8 | Assay conditions for stage III in Table 1 |
| 138 | T52Y; Q53T; I146Q; W183A; N190I; | 18.6 | 86.1 | Assay conditions for stage III in Table 1 |
| 140 | T52Y; Q53T; I146Q; W183S; | 15.9 | 10.8 | Assay conditions for stage III in Table 1 |
| 142 | T52Y; Q53T; I146Q; W183S; N190I; | 24.2 | 50.4 | Assay conditions for stage III in Table 1 |
| 144 | T52Y; Q53T; R126L; W183A; | 9.7 | 43.0 | Assay conditions for stage III in Table 1 |
| 146 | T52Y; Q53T; R126L; W183A; N190I; | 16.1 | >100 | Assay conditions for stage III in Table 1 |
| 148 | T52Y; Q53T; R126L; W183S; | 14.6 | 48.4 | Assay conditions for stage III in Table 1 |
| 150 | T52Y; Q53T; R126L; W183S; N190I; | 22.4 | 89.3 | Assay conditions for stage III in Table 1 |
| 152 | T52Y; Q53T; R126L; I146Q; W183A; | 7.1 | 50.1 | Assay conditions for stage III in Table 1 |
| 154 | T52Y; Q53T; R126L; I146Q; W183A; N190I; | 11.8 | 72.9 | Assay conditions for stage III in Table 1 |
| 156 | T52Y; Q53T; R126L; I146Q; W183S; | 9.3 | 30.2 | Assay conditions for stage III in Table 1 |
| 158 | T52Y; Q53T; R126L; I146Q; W183S; N190I; | 16.7 | 52.7 | Assay conditions for stage III in Table 1 |

TABLE 9
Performance of Transaminase SEQ ID NO: 130

| | | | | | | | Residual |
|---|---|---|---|---|---|---|---|
| | | | | | | | activity |
| | | | | | | | after |
| | | | | | | | heat |
| | | Enzyme | | Reaction | | | treatment |
| | Substrate | powder | IPM | applicable | Product | | at 65, |
| Transaminase | concentration | dosage | tolerance | solvent | dr value | Yield | 24 h |
| SEQ NO | 100 g/L | 5 g/L | >2M | DMSO:ACN = | >100 | 96% | 83% |
| ID: 130 | | | | 35%:15% | | | |

In the paper “Practical Asymmetric Synthesis of a Calcitonin Gene-Related Peptide (CGRP) Receptor Antagonist Ubrogepant”, it is disclosed that the product dr values of transaminases ATA-412 and ATA-426 at a substrate loading of 50 g/L were 61. It can be seen that the engineered transaminase polypeptide developed by the present invention has better activity, selectivity, stability (including thermal stability), solvent tolerance, and substrate tolerance, etc. The solubility of substrate S1 in aqueous/ACN system is much higher than that in aqueous/DMSO system. In order to avoid the damage of enzyme activity by high concentration of ACN as single cosolvent and to improve the solubility of substrate S1 in reaction system, the present invention creatively adopts the mixture of ACN and DMSO as the reaction cosolvent. It is also proved that the hydrolysis of substrate S1 in the mixed-cosolvent system is very effectively controlled, so the engineered transaminase polypeptide disclosed in the present invention is more suitable for industrial production.

Polynucleotides, Control Sequences, Expression Vectors and Host Cells that can be Used to Prepare Engineered Transaminase Polypeptides

In another aspect, the present disclosure provides polynucleotides encoding the engineered polypeptides having transaminase activity described herein. The polynucleotides can be linked to one or more heterologous regulatory sequences that control gene expression to produce a recombinant polynucleotide capable of expressing the polypeptide. Expression constructs comprising heterologous polynucleotides encoding engineered transaminases may be introduced into suitable host cells to express the corresponding engineered transaminase polypeptides.

As apparent to those skilled in the art, the availability of protein sequences and knowledge of codons corresponding to various amino acids provides an illustration of all possible polynucleotides that encode the target protein sequence. The degeneracy of the genetic code, in which the same amino acids are encoded by selectable or synonymous codons, allows for the production of an extremely large number of polynucleotides, all of which encode the improved transaminase polypeptides disclosed herein. Thus, upon determination of a particular amino acid sequence, one skilled in the art can generate any number of different polynucleotides by merely modifying one or more codons in a manner that does not alter the amino acid sequence of the protein. In this regard, the present disclosure particularly contemplates each and every possible alteration of a polynucleotide that can be made by selecting combinations based on possible codon selections, for any polypeptide disclosed herein, comprising those amino acid sequences of exemplary engineered polypeptides provided in Table 6, Table 8 and in the sequence list incorporated herein by reference as SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 100, 102, 104 106, 108, 110, 112, 114, 116, 118, 120, 122, 124, 126, 128, 130, 132, 134, 136, 138, 140, 142, 144, 146, 148, 150, 152, 154, 156, 158.

In various embodiments, the codons are preferably selected to accommodate the host cell in which the protein is produced. For example, codons preferred for bacteria are used to express genes in bacteria; codons preferred for yeast are used to express genes in yeast; and codons preferred for mammals are used for gene expression in mammalian cells.

In some embodiments, the polynucleotides encode a transaminase polypeptides comprising amino acid sequences that have at least about 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146, 148 150, 152, 154, 156, 158, wherein the polypeptides have transaminase activity and one or more of the improved properties described herein, such as the ability to convert compound S1 to products T1 and T2 with increased activity and/or selectivity compared to the polypeptide of SEQ ID NO: 2.

In some embodiments, the polynucleotides encode an engineered transaminase polypeptides comprising amino acid sequences having a percentage of identity described above and having one or more amino acid residue differences as compared to SEQ ID NO: 2. In some embodiments, the present disclosure provides engineered polypeptides having at least 90% sequence identity to the reference sequence of SEQ ID NO: 2 with residue differences that are selected from the following positions: X52, X53, X115, X126, X146, X183, X190, wherein the engineered polypeptides have transaminase activity.

In some embodiments, the polynucleotides encode an engineered transaminase polypeptides comprising amino acid sequences having a percentage of identity described above and having one or more amino acid residue differences as compared to SEQ ID NO: 2. In some embodiments, the present disclosure provides engineered polypeptides having at least 90% sequence identity to the reference sequence of SEQ ID NO: 2 with one or more residue differences selected from: X52Y, X53T, X53K, X53F, X53E, X53H, X115G, X115E, X126L, X146Q, X183A, X183S. X183T, X190L and X190I; wherein the engineered polypeptides converts S1 to IT or L1 with catalytic activity, stability and/or stereoselectivity superior to those of SEQ ID NO: 2.

In some embodiments, the polynucleotides encoding the engineered transaminase polypeptides comprises a polynucleotide selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157.

In some embodiments, the polynucleotides encode polypeptides as described herein, but at a nucleotide level, the polynucleotides have about 50%, 55%, 60%, 65%, 70%, 75%, 80%, 85%, 86%, 87%, 88%, 89%, 90%, 91%, 92%, 93%, 94%, 95%, 96%, 97%, 98%, or 99% or more sequence identity to reference polynucleotides encoding engineered transaminase polypeptides as described herein.

In some embodiments, the reference polynucleotides are selected from SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 7173, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157.

The isolated polynucleotides encoding engineered transaminase polypeptides can be manipulated to enable the expression of the engineered polypeptides in a variety of ways, which comprises further modification of the sequences by codon optimization to improve expression, insertion into suitable expression elements with or without additional control sequences, and transformation into host cells suitable for expression and production of the polypeptide. Depending on the expression vector, manipulation of the isolated polynucleotide prior to insertion of the isolated polynucleotide into the vector may be desirable or necessary. Techniques for modifying polynucleotides and nucleic acid sequences using recombinant DNA methods are well known in the art. Guidance is provided below: Sam brook et al, 2001, Molecular Cloning: A Laboratory Manual, Third Edition, Cold Spring Harbor Laboratory Press; and Current Protocols in Molecular Biology, Ausubel F., Greene Pub. Associates, 1998, updated in 2010.

In another aspect, the present disclosure also relates to recombinant expression vectors, depending on the type of host they are to be introduced into, including a polynucleotide encoding an engineered transaminase polypeptide or variant thereof, and one or more expression regulatory regions, such as promoters and terminators, origin of replication and the like. Alternatively, the nucleic acid sequences of the present disclosure can be expressed by inserting the nucleic acid sequence or the nucleic acid construct comprising the sequence into an appropriate expression vector. In generating the expression vector, the coding sequence is located in the vector such that the coding sequence is linked to a suitable control sequence for expression. The recombinant expression vector can be any vector (e.g., plasmid or virus) that can be conveniently used in recombinant DNA procedures and can result in the expression of a polynucleotide sequence. The choice of vector will generally depend on the compatibility of the vector with the host cells to be introduced into. The vector may be a linear or closed circular plasmid. The expression vector may be an autonomously replicating vector, i.e., a vector that exists as an extrachromosomal entity whose replication is independent of chromosomal replication such as plasmids, extrachromosomal elements, microchromosomes, or artificial chromosomes. The vector may contain any tools for ensuring self-replication. Alternatively, the vector may be a vector that, when introduced into a host cell, integrates into the genome and replicates with the chromosome into which it is integrated. Moreover, a single vector or plasmid or two or more vectors or plasmids that together contain the total DNA to be introduced into the genome of host cell may be used. Many expression vectors useful for embodiments of the present disclosure are commercially available. Exemplary expression vectors can be prepared by inserting a polynucleotide encoding an engineered transaminase polypeptide into plasmid pACYC-Duet-1 (Novagen).

In another aspect, the present disclosure provides host cells comprising a polynucleotide encoding improved transaminase polypeptides of the present disclosure. The polynucleotide is linked to one or more control sequences for expression of transaminase polypeptides in the host cell. Host cells for expression of polypeptides encoded by the expression vectors of the present disclosure are well known in the art, including, but not limited to, bacterial cells such as Escherichia coli, Arthrobacter spp. KNK168, Streptomyces spp. and Salmonella typhimurium cells; fungal cells such as yeast cells (e.g., Saccharomyces cerevisiae or Pichia pastoris); insect cells such as Drosophila S2 and Spodoptera Sf9 cells; animal cells such as CHO, COS, BHK, 293 and Bowes melanoma cells; and plant cells. Exemplary host cells are E. coli BL21 (DE3). The above host cells may be wild-type or engineered cells through genomic edition, such as knockout of the wild-type transaminase gene carried in the host cell's genome. Suitable media and growth conditions for the above host cells are well known in the art.

Polynucleotides for the expression of transaminases can be introduced into cells by a variety of methods known in the art. Techniques include, among others, electroporation, bio-particle bombardment, liposome-mediated transfection, calcium chloride transfection, and protoplast fusion. Different methods of introducing polynucleotides into cells are obvious to those skilled in the art.

### Process of Producing an Engineered Transaminase Polypeptides

When the sequence of an engineered polypeptide is known, the encoding polynucleotide may be prepared by standard solid phase methods according to known synthetic methods. In some embodiments, fragments of up to about 100 bases may be synthesized individually and then ligated (e.g., by enzymatic or chemical ligation methods or polymerase-mediated methods) to form any desired contiguous sequence. For example, the polynucleotides and oligonucleotides of the present disclosure may be prepared by chemical synthesis using, for example, the classic phosphoramidite methods described by Beaucage et al, 1981, Tet Lett 22:1859-69, or Matthes et al., 1984, EMBO J. 3:801-05, as typically practiced in automated synthetic methods. According to the phosphoramidite method, oligonucleotides are synthesized, purified, annealed, ligated, and cloned into a suitable vector, for example, in an automated DNA synthesizer. In addition, essentially any nucleic acid is available from any of a variety of commercial sources.

In some embodiments, the present disclosure further provides a process for preparing or producing an engineered transaminase polypeptide, wherein the process comprises culturing a host cell capable of expressing a polynucleotide encoding the engineered polypeptide under culture conditions suitable for expression of the polypeptide.

In some embodiments, the process of preparing the polypeptide further comprises isolating the polypeptide. The engineered polypeptides may be expressed in suitable cells and isolated (or recovered) from the host cells and/or culture medium using any one or more of the well-known techniques for protein purification, the techniques for protein purification include, among others, lysozyme treatment, sonication, filtration, salting out, ultracentrifugation, and chromatography.

### Methods of Using Engineered Transaminases and Compounds Prepared Therewith

In another aspect, the improved engineered transaminase polypeptides described herein convert pre-chiral compounds of ketone acceptor to chiral amine compounds in the presence of an amino donor. The present disclosure also provides methods for preparing a broad range of compounds I or structural analogs thereof using the engineered transaminase polypeptides disclosed herein. In some embodiments, the engineered transaminase polypeptides may be used in processes for preparing compounds of structural formula I.

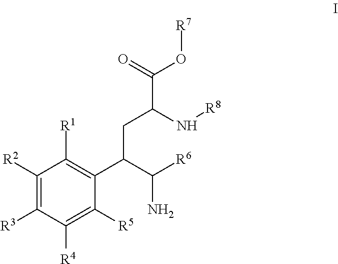

wherein the groups R¹, R², R³, R⁴, R⁵, can be optionally substituted or unsubstituted —H, C₁-C₆ hydrocarbon group, halogen (e.g. —F, —Cl, —Br, —I), —NO₂, —NO—NO, —SO₂R′ or —SOR′, —SR′, —NR′R′, —OR′, —CO₂R′ or —COR′, —C(O)NR′—C(O)NR′, —SO₂NH₂ or —SONH₂, —CN, CF₃; R⁶ can be a C₁-C₆ hydrocarbon group, C₁-C₆ haloalkyl, C₁-C₆ hydroxy-substituted hydrocarbon; R⁷ can be C₁-C₆ hydrocarbon group, C₁-C₆ haloalkyl, C₁-C₆ hydroxy-substituted hydrocarbon; R⁸ can be CBZ protecting group, BOC protecting group, Fomc protecting group, Bn protecting group, methyl(ethyl)oxycarbonyl protecting group. wherein each R′ is independently selected from —H or C₁-C₄ hydrocarbon group.

The amine product shown in structural formula I can be one of, or a mixture of the chiral amine isomers shown in structural formulae II-VI.

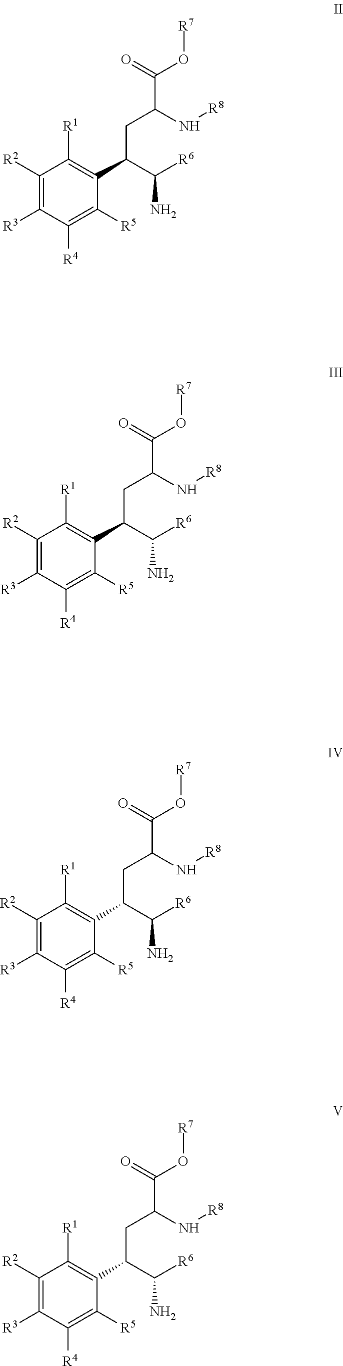

Depending on the activity of ester groups in the structure of compound I, under suitable reaction conditions, such as suitable temperature, pH and solvent conditions, some of the amine products shown in structural formula I can spontaneously form rings to produce lactams shown in structural formula VI.

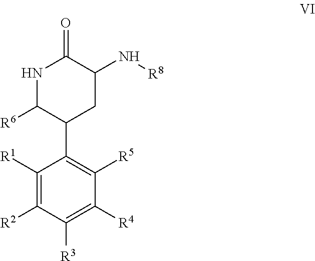

The amine product shown in structural formula VI can be one of, or a mixture of the following chiral amine isomers shown in structural formulae VII-X.

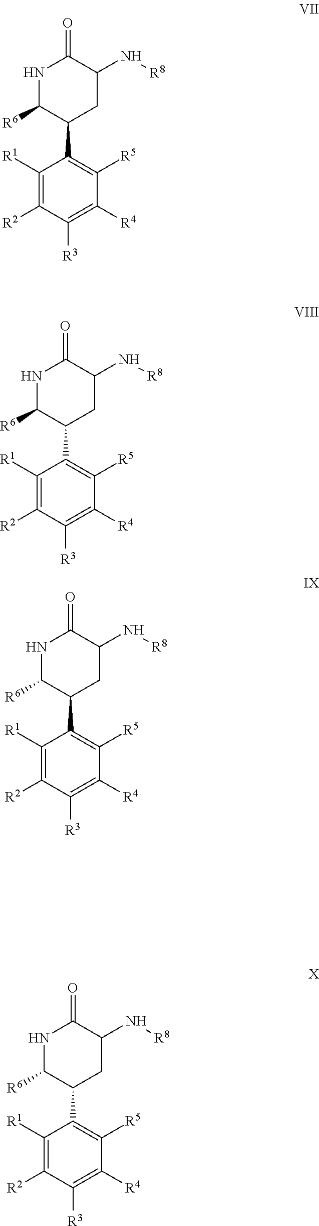

The corresponding substrate structural formula XI that can be catalyzed by transaminases to produce the chiral amine products shown in structural formula I-X is:

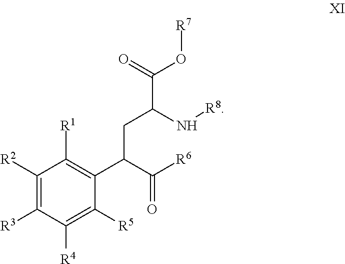

Preferably, the engineered transaminase polypeptide disclosed herein has significant activity to the substrate S1, which has the following structural formula shown below.

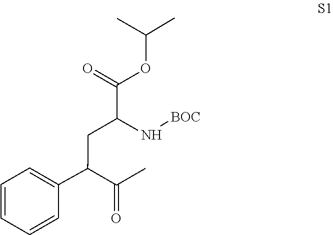 [SMILES: CC(=O)C(CC(NC(=O)OC(C)(C)C)C(=O)OC(C)C)c1ccccc1]

S1 may contain four different isomers ST1, ST2, SD1 or SD2 as follows.

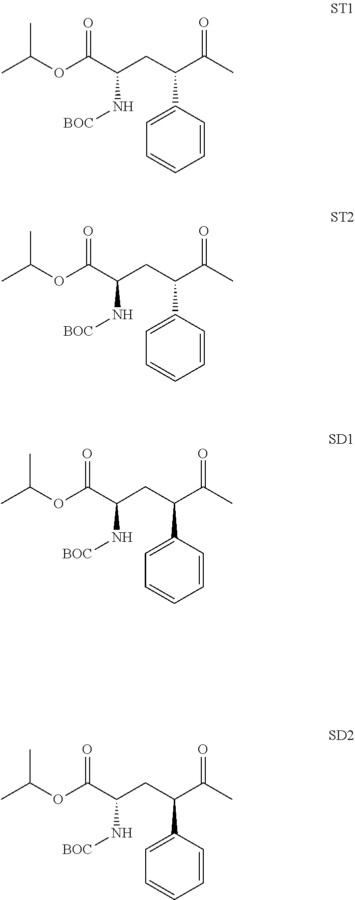

The engineered transaminase polypeptide disclosed in the present invention can convert S1 to I1.

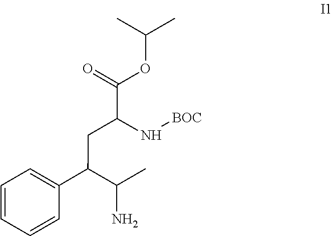 [SMILES: CC(C)OC(=O)C(CC(c1ccccc1)C(C)N)NC(=O)OC(C)(C)C]

I1 may contain four different isomers IT1, IT2, ID1 or ID2 as follows.

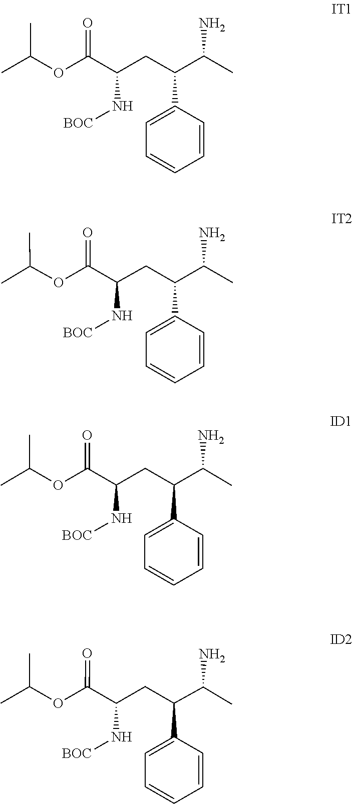

The compounds shown as structural formula IT represent IT1 and/or IT2.

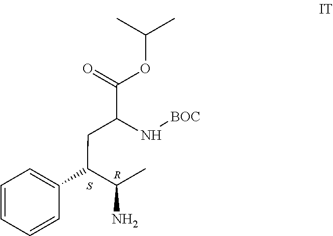

The ester bonds on the I1 structure can spontaneously break and a ring structure forms, resulting in the formation of the compounds shown as T1, T2, D1 or D2.

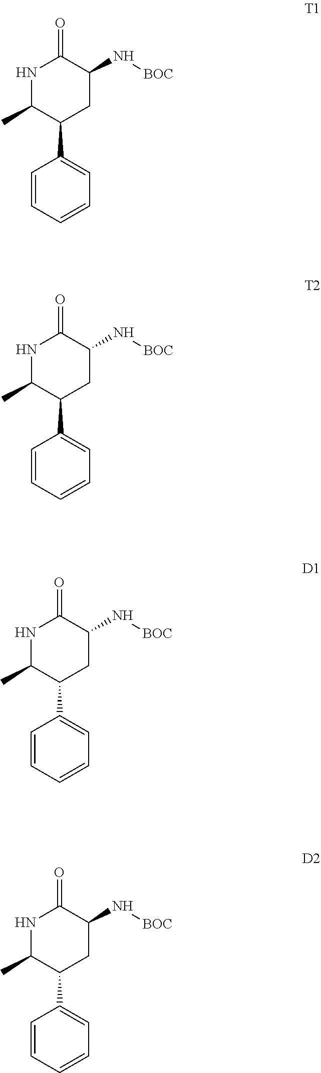 [SMILES: C[C@H]1NC(=O)[C@@H](NC(=O)OC(C)(C)C)C[C@H]1c1ccccc1 ; C[C@H]1NC(=O)[C@H](NC(=O)OC(C)(C)C)C[C@H]1c1ccccc1 ; C[C@H]1NC(=O)[C@H](NC(=O)OC(C)(C)C)C[C@@H]1c1ccccc1 ; C[C@H]1NC(=O)[C@@H](NC(=O)OC(C)(C)C)C[C@@H]1c1ccccc1]

Among the products generated from substrate S1 catalyzed by the engineered transaminase polypeptide disclosed in the present invention, the products in excess are IT1 and IT2 and subsequently T1 and T2.T1 and T2 are represented by the structural formula shown as L1.

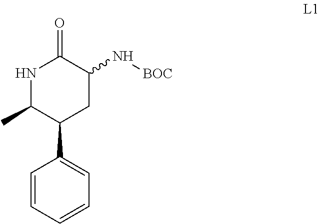 [SMILES: C[C@H]1NC(=O)C(NC(=O)OC(C)(C)C)C[C@H]1c1ccccc1]

The engineered transaminase polypeptide with improved properties described herein converts S1 to one or more product isomers selected from T1, T2, D1, and D2 in the presence of an amino donor. In some embodiments, the dr value of the product (i.e., [T1+T2]/[D1+D2]) is at least 1, 2, 3, 5, 10, 20, 30, 40, 50, 60, 70, 80, 90, 100, or more.

In some embodiments, the engineered transaminase polypeptide used in the above processes may comprise a polypeptide selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60 62, 64, 66, 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142 144, 146, 148, 150, 152, 154, 156, 158, and may also comprise the amino acid sequence having at least 80%, 85%, 86%, 87%, 88%, 89%, 90%, 91%, 92%, 93%, 94%, 95%, 96%, 97%, 98%, 99% or more sequence identity to any one of the reference amino acid sequences selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54 56, 58, 60, 62, 64, 66, 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158.

As described herein and exemplified in embodiments, the present disclosure contemplates a range of suitable reaction conditions that may be used in the process herein, including but not limited to, a range of pH, temperature, buffer, solvent system, substrate loading, polypeptide loading, and reaction time. Additional suitable reaction conditions for performing methods for enzymatically converting a substrate compound to a product compound using the engineered transaminase polypeptide described herein may be readily optimized by routine experiments, which including but not limited to that the engineered transaminase polypeptide is contacted with the substrate compound under experimental reaction conditions of varying concentration, pH, temperature, solvent conditions, and the product compound is detected, for example, using the methods described in the Examples provided herein.

As described above, an engineered polypeptide having transaminase activity for use in the process of the present disclosure generally comprises amino acid sequences that having at least 80%, 85%, 86%, 87%, 88%, 89%, 90%, 91%, 92%, 93%, 94%, 95%, 96%, 97%, 98%, 99% or more sequence identity of any one of the reference amino acid sequences selected from SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58 60, 62, 64, 66, 68, 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140 142, 144, 146, 148, 150, 152, 154, 156, 158.

The substrate compounds in the reaction mixture can be varied, taking into consideration of, for example, the amount of desired product compound, the effect of the substrate concentration on the enzyme activity, the stability of the enzyme under reaction conditions, and the percentage conversion of substrate to product. In some embodiments of the described process, suitable reaction conditions include at least about 0.5 g/L, at least about 1 g/L, at least about 5 g/L, at least about 10 g/L, at least about 15 g/L, at least about 20 g/L, at least about 30 g/L, at least about 50 g/L, at least about 75 g/L, at least about 100 g/L, or even more loadings of substrate S1. The values of the substrate loadings provided herein are based on the molecular weight of compound S1, it is also anticipated that the equivalent molar amounts of various hydrates and salts of compound may also be used in the process.

In the process described herein, the engineered transaminase polypeptide catalyzes the formation of a chiral amine product from a ketone substrate with an amino donor. In some embodiments, the amino donor in the reaction conditions comprises any suitable amino acid selected from alanine, isopropylamine (also referred to as 2-aminopropane), phenylalanine, glutamine, leucine, or 3-aminobutyric acid, or includes any suitable chiral amine or non-chiral amine selected from methylbenzylamine; the amino donor may also be in the form of a salt (e.g., alanine hydrochloride, alanine acetate, isopropylamine hydrochloride, isopropylamine acetate, etc.) applied in embodiments. In some embodiments, the amino donor is isopropylamine. In some embodiments, suitable reaction conditions include the presence of the amino donor, in particular isopropylamine, at a loading of at least one times of the molar loading of the substrate S1. In some embodiments, isopropylamine is present at a loading of 0.1 M to about 4.0 M.

In embodiments of the reaction process, the reaction conditions may include a suitable pH. As described above, the desired pH or desired pH range may be maintained by using an acid or base, a suitable buffer, or a combination of buffering and addition of an acid or base. The pH of the reaction mixture may be controlled before and/or during the reaction process. In some embodiments, suitable reaction conditions include a solution pH of about 7 to about 11.5. In some embodiments, the reaction conditions include a solution pH of about 7, 7.5, 8, 8.5, 9, 9.5, 10, 10.5, 11, 11.5.

In embodiments of the process herein, suitable temperatures may be used for the reaction conditions, taking into consideration of, for example, the increase in reaction rate at higher temperatures, the activity of the enzyme for sufficient duration of the reaction. Accordingly, in some embodiments, suitable reaction conditions include a temperature of about 10° C. to about 65° C., about 25° C. to about 50° C., about 25° C. to about 40° C., or about 25° C. to about 30° C. In some embodiments, a suitable reaction temperature comprises a temperature of about 25° C., 30° C., 35° C., 40° C., 45° C., 50° C., 55° C., 60° C., or 65° C. In some embodiments, the temperature during the enzymatic reaction may be maintained at a certain temperature throughout the reaction. In some embodiments, the temperature during the enzymatic reaction may be adjusted over a temperature profile during the course of the reaction.

The process of using engineered transaminases are generally performed in water or solvents. Suitable solvents include aqueous buffer solutions, organic solvents, and/or co-solvent systems, which generally include aqueous and organic solvents. The aqueous solution (water or aqueous co-solvent system) may be pH-buffered or unbuffered. In some embodiments, the process of using the engineered transaminase polypeptide is generally performed in an aqueous co-solvent system comprising: an organic solvent (e.g., methanol, ethanol, propanol, isopropyl alcohol (IPA), dimethyl sulfoxide (DMSO), dimethyl formamide (DMF), isopropyl acetate, ethyl acetate, butyl acetate, 1-octanol, heptane, octane, methyl tert-butyl ether (MTBE), toluene, etc.), ionic liquids (e.g., 1-ethyl 4-methylimidazole tetrafluoroborate, 1-butyl-3-methylimidazole tetrafluoroborate, 1-butyl-3-methylimidazole hexafluorophosphate, etc.). The organic solvent component of the aqueous co-solvent system may be miscible with the aqueous component, providing a single liquid phase, or may be partially miscible or immiscible with the aqueous component, providing two liquid phases. The carbon dioxide generated during the transamination reaction may cause foam formation, and antifoam agents may be added as appropriate. Exemplary aqueous co-solvent systems contain water and one or more organic solvents. In general, the organic solvent component of the aqueous co-solvent system is selected such that it does not completely inactivate the transaminase. Suitable co-solvent systems can be readily identified by measuring the enzymatic activity of a particular engineered transaminase with a defined substrate of interest in a candidate solvent system, utilizing enzymatic activity assay such as those described herein. In some embodiments of the process, suitable reaction conditions include an aqueous co-solvent, which comprising from about 1% to about 100% (v/v), from about 1% to about 60% (v/v), from about 2% to about 60% (v/v), from about 5% to about 60% (v/v), from about 10% to about 60% (v/v), from about 10% to about 50% (v/v), or from about 10% to about 40% (v/v) concentration of the solvent mixture of DMSO and ACN. In some embodiments of the process, suitable reaction conditions include a solvent mixture containing at least about 1%, 5%, 10%, 15%, 20%, 25%, 30%, 35%, 40%, 50%, 60% concentrations of the solvent mixture of DMSO and ACN.

Suitable reaction conditions may include combinations of reaction parameters that provide for the biocatalytic conversion of the substrate compound to its corresponding product compound. Accordingly, in some embodiments of the process, the combination of reaction parameters includes (a) a loading of about 10 g/L to 100 g/L of substrate S1; (b) about 1 g/L to 50 g/L of the engineered polypeptide; (c) a loading of about 0.1 M to 4.0 M of isopropylamine; (d) a pH of about 7.0 to 11.5; (e) a temperature of about 10° C. to 65° C. and (f) 1%-70% of the solvent mixture of DMSO and ACN.

In carrying out the enzyme-catalyzed reactions described herein, the engineered polypeptide may be added to the reaction mixture in the form of partially purified or purified enzyme, heat-treated enzyme solution, whole cells transformed with the gene encoding the enzyme polypeptide, and/or as cell extracts and/or lysates of such cells. Whole cells transformed with the genes encoding the engineered polypeptides, or cell extracts thereof, lysates thereof, and isolated enzymes can be used in a variety of different forms, including solid (e.g., lyophilized, spray-dried, etc.) or semi-solid (e.g., a crude pastes). The cell extracts or cell lysates may be partially purified by precipitation (e.g., ammonium sulfate, polyethyleneimine, heat treatment, or the like) followed by a desalting procedure (e.g., ultrafiltration, dialysis, and the like) prior to lyophilization. Any enzyme product can be cross-linked or immobilized to a solid-phase material (e.g., resin) by using a known cross-linking agent such as, for example, glutaraldehyde.

In some embodiments of the enzyme-catalyzed reactions described herein, the reactions are carried out under suitable reaction conditions as described herein, wherein the engineered polypeptide is immobilized to a solid support. Solid supports useful for immobilizing the engineered polypeptide for carrying out the enzyme-catalyzed reactions include, but are not limited to, beads or resins, such as polymethacrylate with epoxy functional group, polymethacrylate with amino-epoxy functional group, styrene/DVB copolymer or polymethacrylate with octadecyl functional group. Exemplary solid supports include, but are not limited to, chitosan beads, EupergitC, and SEPABEAD (Mitsubishi), including the following different types of SEPABEAD: EC-EP, EC-HFA/S, EXA252, EXE119, and EXE120.

In some embodiments, wherein the engineered polypeptide may be expressed in the form of a secreted polypeptide, a culture medium containing the secreted polypeptide may be used in the process herein.

In some embodiments, the solid reactants (e.g., enzymes, salts, etc.) may be provided to the reaction in a variety of different forms, including powders (e.g., lyophilized, spray-dried, etc.), solutions, emulsions, suspensions, etc. The reactants can be readily lyophilized or spray dried using methods and instrumentation commonly known to one skilled in the art. For example, the protein solutions can be frozen in small aliquots at −80° C. and then added to a pre-chilled lyophilization chamber, followed by the application of a vacuum.

In some embodiments, there are various options for the order or manner in which the reactants are added. The reactants may be added together to the solvent (e.g., monophasic solvent, biphasic aqueous co-solvent system, etc.); or alternatively, some reactants may be added first and others may be added flow-through or in batch intervals.

Different features and embodiments of the present disclosure are exemplified in the following representative embodiments, which are intended to be illustrative and not restrictive.

### DRAWINGS

FIG. 1 Synthetic route of Ubrogepant.

FIG. 2 Reaction catalyzed by transaminase of the present invention.

FIG. 3 General workflows of virtual screening.

FIG. 4 Distribution of amino acid sites near substrate ST1.

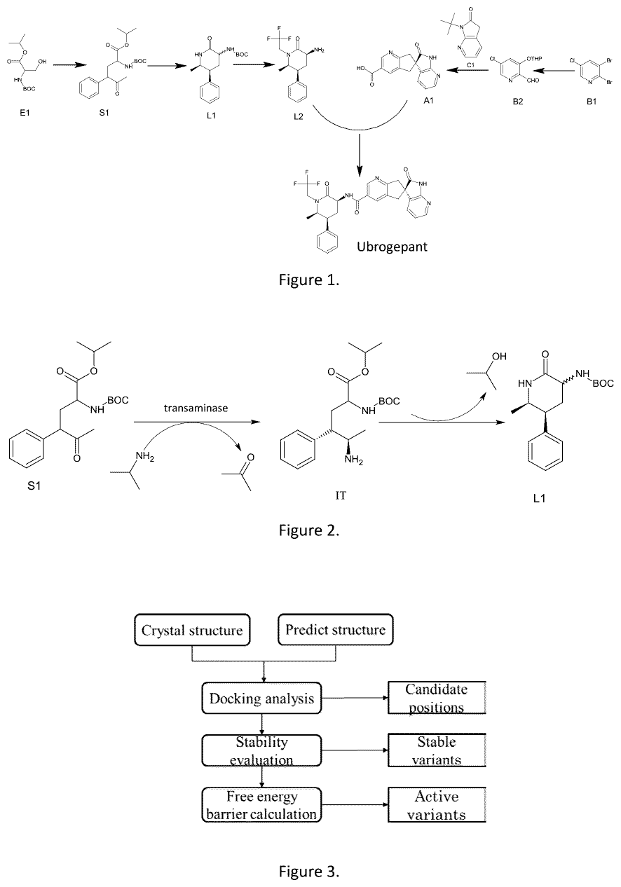

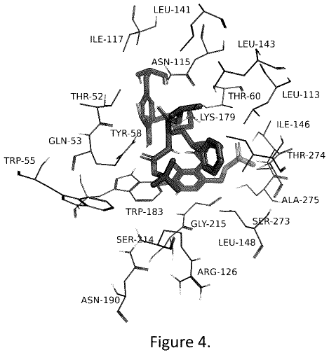

### EXAMPLES

### Example 1 Screening of Transaminases in Stage I

200 μL of cell lysis buffer (containing 1 g/L lysozyme, 0.5 g/L PMBS, 0.5 g/L nuclease, dissolved in sodium tetraborate buffer, pH 10.5) was added to each well of microtiter plates containing different transaminase wet cells, and the plates were shaken for 1 h to obtain cell lysate. The lysate was centrifuged, and the supernatant was transferred to a new deep-well plate to obtain the enzyme solution available for the assay reaction. 40 μL of 20 g/L stock solution of substrate dissolved in DMSO, 50 μL of reaction mixture (containing 4 M isopropylamine, 2 g/L PLP, dissolved in sodium tetraborate buffer, pH adjusted to 10.5 (40° C.) with concentrated hydrochloric acid), and 110 μL of enzyme solution of each transaminase were added sequentially to the 96-well deep-well plate. The final concentration of each component of the reaction system is [5 g/L substrate, 55% enzyme solution (v/v), 20% DMSO, 0.5 g/L PLP, 1M isopropylamine, 0.025M sodium tetraborate buffer, pH 10.5], and the plates were placed in a temperature-constant shaker at 45° C. for 24 h. After the reaction, the plates were removed from the shaker and heated in a water bath shaker at 70° C. for 1 h, after which neat acetonitrile was added at 1:1 volume ratio to fully quench the reaction. The samples of quenched reactions were diluted to 2.5 g/L for HPLC analysis, and among the screened transaminase variants, SEQ ID NO:2 showed best activity and selectivity with a yield of 29% and a dr value of 0.3.

### Example 2 Expression of Transaminase Polypeptide

A single colony of E. coli BL21 (DE3) with the expression plasmid of target transaminase polypeptide was inoculated into a 250 mL conical flask containing 50 mL LB medium (containing 30 μg/mL chloramphenicol), and it was cultured in a shaking incubator overnight at 30° C. When the OD₆₀₀ of the culture medium reached 2, the culture was subcultured into a 1000 mL conical flask containing 250 mL of TB medium at 5% (v/v) inoculum and incubated at 30 in a shaking incubator. When the OD₆₀₀ of the TB culture medium reached 0.6, IPTG was added to induce the expression of transaminase at a final concentration of 1 mM IPTG. After expression for 20 h, the culture was centrifuged (8000 rpm, 10 min), the supernatant was discarded after centrifugation, and the cells were collected to obtain wet cells. The wet cells were used directly in the preparation of enzyme solution or could be stored frozen at −20° C. until use.

### Example 3 Solvent Tolerance Test for SEQ ID NO. 2

0.5 g of SEQ ID NO: 2 wet cells prepared using the procedures as described in Example 2 was added to 5 mL of cell lysis buffer (containing 1 g/L lysozyme, 0.5 g/L PMBS, 0.5 g/L nuclease, dissolved in sodium tetraborate buffer, pH 10.5), and it was shaken for 1 h to break the cells to obtain cell lysate. The cell lysate was centrifuged and the supernatant was collected to obtain enzyme solution. The reactor was preheated to 45. Substrate S1 dissolved with DMSO, methanol or IPA respectively, reaction mixture (containing 4M isopropylamine, 2 g/L PLP, dissolved in sodium tetraborate buffer, pH 10.5 (40) adjusted with concentrated hydrochloric acid), sodium tetraborate buffer (pH 10.5), and enzyme solution of SEQ ID NO:2, were added sequentially to the reaction flask with magnetic stirring at 400 rpm. The final concentrations of the reaction system was [5 g/L substrate, 20% enzyme solution (v/v), 20% cosolvent (v/v), 0.5 g/L PLP, 1 M isopropylamine, 0.025 M sodium tetraborate buffer, pH 10.5]. After the reaction for 24 h, the reactor was warmed up to 70° C. and maintained at this temperature for 1 h. After that, 5 mL of neat acetonitrile was added to quench the reaction, and the sample was taken for HPLC analysis. The results of the reaction with methanol, DMSO or isopropanol are shown as follows.

| Cosolvent | Reaction yield | dr |
|---|---|---|
| Methanol | 86.7% | 1.7 |
| DMSO | 60.2% | 0.3 |
| Isopropanol | 64.5% | 0.3 |

### Example 4 Expression of Enzyme Mutant Library and Preparation of Enzyme Solution for Screening

Mutant colonies were picked from the LB agar plates, inoculated into LB medium (containing chloramphenicol) in a 96-well shallow plate and cultured overnight at 30° C. in a shaker. When OD₆₀₀ of the culture reached 2˜3, 20 μL of the above culture was taken and inoculated into a TB medium with chloramphenicol in a 96-well deep plate (400 μL TB medium per well) and cultured at 30. When OD₆₀₀ of deep-well culture reached 0.6-0.8, IPTG was added to induce expression at a final concentration of 1 mM, and the expression undertook at 30° C. overnight (18-20 h). Once the overnight expression was done, the culture was centrifuged, and the supernatant was removed to obtain wet cell pellets, which was stored in the refrigerator at −20° C. for at least 24 h. Then, 200 μL cell lysis buffer (containing 1 g/L lysozyme, 0.5 g/L PMBS, 0.5 g/L nuclease dissolved in 0.05 M sodium tetraborate buffer, pH 10.5) was added to the cell pellets in each well of the plate, and the plate was shaken for 1 h to break the cell to obtain cell lysate. The cell lysate was centrifuged and the supernatant was transferred to a new deep-well plate to obtain an enzyme solution that could be used for the screening assays.

### Example 5 High Throughput Screening of Enzyme Variants in Stage II

Into a deep-well plate, 70 μL of stock solution of substrate dissolved in methanol, 40 μL of sodium tetraborate buffer (pH 10.5), 50 μL of isopropylamine mixture (containing 4 M isopropylamine, 2 g/L PLP, dissolved in sodium tetraborate buffer, pH adjusted to 10.5 (40° C.) with concentrated hydrochloric acid), and 60 μL of enzyme solution prepared as in Example 4 were added, making the final concentration of each component of the reaction system as of [5 g/L substrate, 30% enzyme solution (v/v), 35% methanol, 0.5 g/L PLP, 1M isopropylamine, 0.025M sodium tetraborate buffer, pH 10.5]. The plate was shaken at 45° C. for 24 h. After the reaction, the plate was removed from shaker and heated in a water bath shaker at 70 for 1 h. Finally, neat acetonitrile was added at 1:1 volume ratio to fully quench the reaction. The samples of quenched reactions were diluted to 2.5 g/L for HPLC analysis.

### Example 6 Reaction in Mixture Cosolvent of DMSO:ACN

0.5 g of SEQ ID NO:24 wet cells prepared using the procedures described in Example 2 was added to 5 mL of cell lysis buffer (containing 1 g/L lysozyme, 0.5 g/L PMBS, 0.5 g/L nuclease, dissolved in sodium tetraborate buffer, pH 10.5), and it was shaken for 1 h to break the cells to obtain cell lysate. The cell lysate was centrifuged and the supernatant was collected to obtain enzyme solution. The reactor was preheated to 45. Substrate S1 dissolved in methanol, DMSO, 80% DMSO:20% ACN, 70% DMSO:30% ACN, 60% DMSO:40% ACN, 50% DMSO:50% ACN respectively, reaction mixture (containing 4M isopropylamine, 2 g/L PLP, dissolved in sodium tetraborate buffer, adjusted to pH 10.5 (40° C.) with concentrated hydrochloric acid), and SEQ ID NO:24 enzyme solution was added sequentially to the reaction flask with magnetic stirring at 400 rpm. The final concentration of the reaction system was [50 g/L substrate, 25% (v/v) SEQ ID NO:24 enzyme solution, 50% (v/v) cosolvent, 0.5 g/L PLP, 1 M isopropylamine, 0.025 M sodium tetraborate buffer, pH 10.5]. After the reaction for 24 h, the reactor was warmed up to 70° C. and maintained at this temperature for 1 h. After that, 5 mL of neat acetonitrile was added to quench the reaction, and the sample was taken for HPLC analysis. The results of the reactions with different cosolvent systems are shown in the table below.

| Final concentration of | | |
|---|---|---|
| cosolvent (v/v) | Reaction yield | dr |
| 50% Methanol | 23.1% | 7.3 |
| 50% DMSO | 18.9% | 7.1 |
| 40% DMSO: 10% ACN | 21.2% | 7.4 |
| 35% DMSO: 15% ACN | 30.1% | 8.5 |
| 30% DMSO: 20% ACN | 27.4% | 8.3 |
| 25% DMSO: 25% ACN | 26.3% | 8.7 |

### Example 7 High Throughput Screening of Enzyme Variants in Stage III

Into a deep-well plate, 70 μL of stock solution of substrate dissolved in 70% DMSO:30% ACN solvent mixture, 40 μL of sodium tetraborate buffer (pH 10.5), 50 μL of isopropylamine mixture (containing 4 M isopropylamine, 2 g/L PLP, dissolved in sodium tetraborate buffer, pH adjusted to 10.5 (40° C.) with concentrated hydrochloric acid), and 60 μL of enzyme solution prepared as in Example 4 were added, making the final concentration of each component of the reaction system as of [30 g/L substrate, 30% enzyme solution (v/v), 35% DMSO:15% ACN, 0.5 g/L PLP, 1M isopropylamine, 0.025M sodium tetraborate buffer, pH 10.5]. The plate was shaken at 55° C. for 24 h. After the reaction, the plate was removed from shaker and heated in a water bath shaker at 70 for 1 h. Finally, neat acetonitrile was added at 1:1 volume ratio to fully quench the reaction. The samples of quenched reactions were diluted to 5 g/L for sample detection.

### Example 8 Test of Enzyme's Thermal Stability

Enzyme stock solution preparation: 1.5 g of SEQ ID NO: 130 wet cells was dissolved in 30 mL of cell lysis buffer (1 g/L lysozyme, 0.5 g/L PMBS, 0.5 g/L nuclease, dissolved in sodium tetraborate buffer, pH10.5), and it was shaken for 1 h to break the cells to obtain cell lysate. The cell lysate was centrifuged and the supernatant was collected to obtain enzyme solution.

Heat treatment of enzyme solution: 3 mL of the enzyme solution prepared as above was heated at 45, 55 and 65 separately in a water bath for 2 h, 24 h respectively.

Assay of heat-treated enzyme solution: The temperature of reactor was set at 45, the substrate dissolved in 70% DMSO:30% ACN mixture, reaction mixture [containing 4M isopropylamine, 2 g/L PLP, dissolved in sodium tetraborate buffer, adjust the pH to 10.5 (40) with concentrated hydrochloric acid], heat-treated enzyme solution of SEQ ID NO: 130 were added sequentially to the reaction flask with magnetic stirring at 400 rpm. The final concentration of the reaction system is [50 g/L substrate, 35% DMSO:15% ACN, 1 M isopropylamine, 0.5 g/L PLP, 0.025 M sodium tetraborate buffer, pH 10.5, 25% (v/v) SEQ ID NO: 130 enzyme solution]. After the reaction for 24 h, the reactor was warmed to 70 and maintained at this temperature for 1 h. Subsequently, 5 mL of neat acetonitrile was added quench the reaction. The results of reactions using enzyme solutions pretreated at different temperatures were detected by HPLC as shown in the table below.

| | Heat treatment | | Heat treatment | | Heat treatment | |
|---|---|---|---|---|---|---|
| | at 45 | | at 55 | | at 65 | |
| | Yield | dr | Yield | dr | Yield | dr |
| 0 h | 72.6% | >100 | 72.6% | >100 | 72.6% | >100 |
| 2 h | 72.5% | >100 | 72.3% | >100 | 70.9% | >100 |
| 24 h | 72.8% | >100 | 72.5% | >100 | 58.1% | >100 |

### Example 9 Test of Enzyme's pH Tolerance

The reactor was set at 55. The substrate dissolved in 70% DMSO:30% ACN solvent mixture, reaction mixture (containing 4M isopropylamine, 2 g/L PLP, dissolved in sodium tetraborate buffer, pH adjusted to 10.5 (40° C.) with concentrated hydrochloric acid), and SEQ ID NO: 130 enzyme solution were added sequentially to the reaction flask with magnetic stirring at 400 rpm. The final concentrations of the reaction system was [50 g/L substrate, 35% DMSO:15% ACN, 1M isopropylamine (pH 9.5, 10.5, 11, 11.5), 0.5 g/L PLP, 0.025M sodium tetraborate buffer (pH 9.5, 10.5, 11, 11.5), 25% (v/v) SEQ ID NO: 130 enzyme solution]. After the reaction for 24 h, the reactor was warmed up to 70° C. and maintained at this temperature for 1 h. Subsequently, 5 mL of neat acetonitrile was added to quench the reaction. Samples were taken for HPLC analysis. The results of 24 h reactions at pH 9.5, pH 10.5, pH 11 and pH 11.5 conditions are shown in the table below.

| | Reaction yield | dr |
|---|---|---|
| pH 9.5 | 70.7% | >100 |
| pH 10.5 | 88.1% | >100 |
| pH 11 | 52.3% | >100 |
| pH 11.5 | 15.8% | >100 |

### Example 10 Optimization of Reaction Temperature

The temperature of reactors was set at 30° C., 45° C., 55° C. and 65° C., respectively. The substrate dissolved in 70% DMSO:30% ACN solvent mixture, reaction mixture (containing 4M isopropylamine, 2 g/L PLP, dissolved in sodium tetraborate buffer, pH adjusted to 10.5 (40° C.) with concentrated hydrochloric acid), and SEQ ID NO: 130 enzyme solution were added sequentially to the reaction flask with magnetic stirring at 400 rpm. The final concentration of the reaction system was [50 g/L substrate, 35% DMSO:15% ACN solvent mixture, 1M isopropylamine (pH 10.5), 0.5 g/L PLP, 0.025M sodium tetraborate buffer (pH 10.5), 25% (v/v) SEQ ID NO: 130 enzyme solution]. After the reaction for 24 h, the reactor was warmed up to 70° C. and maintained at this temperature for 1 h. Subsequently, 5 mL of neat acetonitrile was added to quench the reaction. Samples were taken for HPLC analysis. The results of 24 h reactions at 30, 45, 55 and 65 are shown in the table below.

| | Reaction yield | dr |
|---|---|---|
| 30 | 52.4% | >100 |
| 45 | 76.2% | >100 |
| 55 | 88.1% | >100 |
| 65 | 66.4% | >100 |

### Example 11 Test for Solvent Tolerance of Engineered Transaminase

The reactor was set at 55. The substrates dissolved in methanol, DMSO, isopropanol, or ACN, respectively, the reaction mixture (containing 4M isopropylamine, 2 g/L PLP, dissolved in sodium tetraborate buffer, pH adjusted to 10.5 (40° C.) with concentrated hydrochloric acid), SEQ ID NO: 130 enzyme solution were added sequentially to the reaction flask with magnetic stirring at 400 rpm. The final concentration of the reaction system was [50 g/L substrate, 20%, 35%, 50% or 60% (v/v) of methanol, DMSO, isopropanol, or ACN, 1 M isopropylamine (pH 10.5), 0.5 g/L PLP, 0.025 M sodium tetraborate buffer (pH 10.5), 25% (v/v) SEQ ID NO: 130 enzyme solution]. After the reaction for 24 h, the reactor was warmed up to 70° C. and maintained at this temperature for 1 h. Subsequently, 5 mL of neat acetonitrile was added to quench the reaction. Samples were taken for HPLC analysis. The results of 24 h reactions under different cosolvent concentration conditions are shown in the table below.

| | Methanol | | DMSO | | Isopropanol | | ACN | |
|---|---|---|---|---|---|---|---|---|
| | Yield | dr | Yield | dr | Yield | dr | Yield | dr |
| 25% | 62.1% | >100 | 42.9% | >100 | 46.4% | >100 | 67.5% | >100 |
| 35% | 69.5% | >100 | 64.4% | >100 | 27.3% | >100 | 32.3% | >100 |
| 50% | 85.3% | >100 | 67.5% | >100 | 14.9% | >100 | 17.2% | >100 |
| 60% | 38.1% | >100 | 21.6% | >100 | 5.2% | >100 | 4.1% | >100 |

### Example 12 Reaction in Neat Organic Condition

The reactor was set at 55. The substrates S1 dissolved in methanol, DMSO, isopropanol, ACN, ethyl acetate, isopropyl acetate, or toluene, respectively, isopropylamine mixture (containing 0.25 mL pure water and 3.5 g/L PLP), SEQ ID NO: 130 wet cells were added sequentially to the reaction flask with magnetic stirring at 400 rpm. The final concentration of the reaction system was [50 g/L substrate, 86% (v/v) of methanol, DMSO, isopropanol, ACN, ethyl acetate, isopropyl acetate, or toluene, 1M isopropylamine, 0.5 g/L PLP, 50 g/L SEQ ID NO: 130 wet cells]. After the reaction for 24 h, the reactor was warmed up to 70° C. and maintained at this temperature for 1 h. Subsequently, 5 mL of neat acetonitrile was added to quench the reaction. Samples were taken for HPLC analysis. The results of reactions using different organic solvents are shown in the table below.

| | Reaction yield | dr |
|---|---|---|
| Methanol | 24.1% | >100 |
| DMSO | 17.4% | >100 |
| Isopropyl alcohol | 36.6% | >100 |
| ACN | 11.4% | >100 |
| Ethyl acetate | 43.2% | >100 |
| Isopropyl Acetate | 38.1% | >100 |
| Toluene | 26.3% | >100 |

### Example 13 Fermentation Process and Downstream Process

A single colony of E. coli BL21 (DE3) containing an expression plasmid bearing the gene for target engineered transaminase peptide was inoculated into 50 mL LB broth (5 0.0 g/L Yeast Extract LP0021, 10 g/L TryptoneLP0042, 10 g/L NaCl) containing 30 μg/mL chloramphenicol, the culture was incubated for 16 hours with shaking at 250 rpm in a 30° C. shaker. When the OD₆₀₀ of the culture reached 3.5 to 4.5, the culture was removed from the shaker and immediately used to inoculate medium in a 1.0 L fermentor with 0.4 L of growth medium pre-sterilized in a 121° C. autoclave for 30 min. Temperature of fermentor was maintained at 37° C. The growth medium in fermentor was agitated at 200-800 rpm and air was supplied to the fermentation vessel at 0.4-0.8 L/min to maintain the dissolved oxygen level at 30% saturation or greater. The culture was maintained at pH 7.0 by addition of 25-28% v/v ammonium hydroxide. Cell growth was maintained by feeding a feed solution containing 500 g/L edible dextrose monohydrate, 12 g/L ammonium chloride and 5 g/L magnesium sulfate heptahydrate. After the OD₆₀₀ of culture reached 25 5, the temperature of fermentor was decreased and maintained at 30° C., and the expression of transaminase polypeptides was induced by the addition of isopropyl-β-D-thiogalactopyranoside (IPTG) to a final concentration of 0.1 mM. Fermentation process then continued for additional 16 hours. After the fermentation process was completed, wet cells were harvested using a Thermo Multifuge X3R centrifuge at 8000 rpm for 10 minutes at 4° C. Harvested wet cells were used directly in the downstream process or stored frozen at −20° C.

In downstream process, 6 g of wet cells was resuspended in 30 mL of 100 mM potassium phosphate buffer containing 250 μM pyridoxal 5′-phosphate (PLP), pH 7.5 at 4° C. The transaminases were released from the cells by pressure crushing at 800 bar for 2 times using a homogenizer. The resulting cell lysate was clarified by centrifugation at 8000 rpm for 10 min at 4° C. using a Thermo Multifuge X3R centrifuge. The clarified supernatant was dispensed into shallow containers, frozen at −20° C., or could be lyophilized to a enzyme powder. The transaminase enzyme powder was stored frozen at −20° C.

### Example 14 Reactions Catalyzed by Transaminase SEQ ID NO: 24 or SEQ ID NO: 130

Two 500 mL reaction flasks were set in a water bath maintained at 55. 20 g substrate S1, 70 mL DMSO and 30 mL ACN were added sequentially to each of the two 500 mL reaction flasks, stirring to dissolve S1 completely. 50 mL reaction mixture (containing 4M isopropylamine, 2 g/L PLP, dissolved in sodium tetraborate buffer and pH adjusted to 10.5 (40° C.) with concentrated hydrochloric acid), SEQ ID NO: 24 or SEQ ID NO: 130 enzyme powder prepared using the procedures described in Example 13, were then added to each reaction flask, and the final concentration of the reaction system was [100 g/L S1, 35% DMSO:15% ACN, 1 M isopropylamine (pH 10.5), 0.5 g/L PLP, 0.025 M sodium tetraborate buffer (pH 10.5), 20 g/L SEQ ID NO: 24 or SEQ ID NO: 130]. The pH of the reaction system was maintained at pH 10.3-pH10.5 with 6M isopropylamine aqueous solution using a real-time pH controller. 200 μL reaction samples were taken at 24 h, 48 h, 72 h and 96 h during the reaction, respectively. The reaction samples were heated at 70 for 1 hour, followed by the addition of 200 μL neat acetonitrile for quenching. The results are shown in the following table.

| | SEQ ID NO: 24 (Reaction 1) | | SEQ ID NO: 130 (Reaction 2) | |
|---|---|---|---|---|
| | Yield | dr | Yield | dr |
| 24 h | 25.7% | 8.1 | 64.4% | >100 |
| 48 h | 47.4% | 8.4 | 96.2% | >100 |
| 72 h | 70.3% | 8.1 | — | — |
| 96 h | 92.1% | 7.9 | — | — |

### Example 15 Reaction Work-Up

The reaction solution of Example 14 was put in a Rotary evaporator at 40° C., −0.095 MPa to remove isopropylamine and acetonitrile, and then the reaction system was adjusted to pH 10 with 2M sodium hydroxide. 100 mL of ethyl acetate was used to extract the reaction, the upper clear layer was removed, and the lower aqueous phase was extracted again with 50 mL of ethyl acetate. The ethyl acetate layers were combined, and it was washed twice with 50 mL of NaCl-saturated water. The resulting liquid was partitioned and separated. After that, the ethyl acetate in product was removed using a Rotary evaporator at 40° C. and −0.095 MPa, and the ethyl acetate was collected by a condensing equipment. The crude product was subsequently crystallized using a mixture of ethyl acetate/n-heptane (1:2 (v/v)) solvent. Reaction 1 finally yielded 13.1 g of pure product with a total product yield of 77.4%, dr value of 79 and purity of 98.5%. Reaction 2 finally yielded 14.3 g of pure product with a total product yield of 84.5%, dr value of >100, and product purity>99%.

1. An engineered transaminase polypeptide comprising an amino acid sequence having at least 90% sequence identity to the reference sequence shown in SEQ ID NO: 2; wherein the polypeptide is capable of converting compound S1 to compound 11.

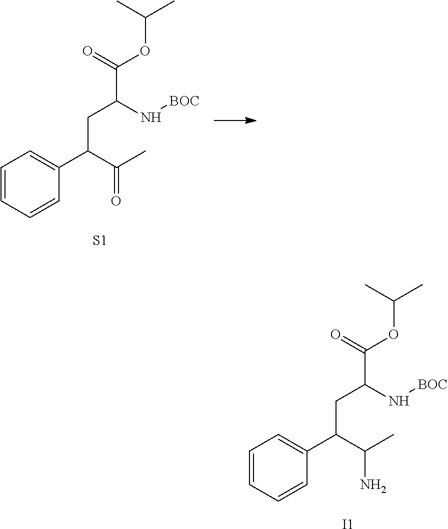

2. The engineered polypeptide of claim 1, wherein the amino acid sequence comprises an amino acid residue difference as compared to SEQ ID NO: 2 at residue position X53 selected from T, K, F, E and H.

3. The engineered polypeptide of claim 2, in which the amino acid sequence further comprises one or more residue differences as compared to SEQ ID NO: 2 selected from: X52Y, X53T, X53K, X53F, X53E, X53H, X115G, X115E, X126L, X146Q, X183A, X183S. X183T, X190L and X190I; the engineered polypeptide converts S1 to IT or L1 with catalytic activity, stability and/or stereoselectivity superior to those of SEQ ID NO: 2.

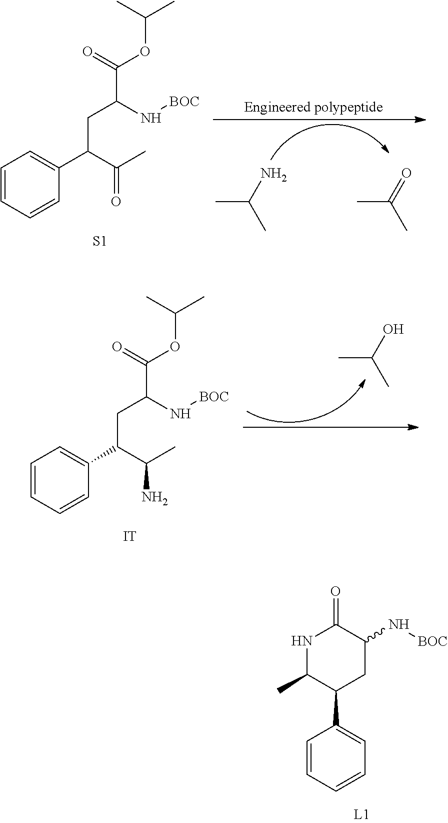

4. The engineered polypeptide of claim 3, in which the amino acid sequence comprises a sequence selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158.

5. A polypeptide immobilized on a solid material by chemical bonding or physical adsorption method, wherein the polypeptide is selected from the transaminase polypeptide of claim 1.

6. A polynucleotide encoding the polypeptide of claim 1.

7. The polynucleotide of claim 6, wherein the polynucleotide sequence is selected from the group consisting of SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157.

8. An expression vector, the vector comprises the polynucleotide of claim 6.

9. The expression vector of claim 8, which comprises a plasmid, a cosmid, a bacteriophage or a viral vector.

10. A host cell, which comprising the expression vector of claim 8, wherein the host cell is preferably E. coli.

11. A method of preparing a transaminase polypeptide, which comprises the steps of culturing the host cell of claim 10 and obtaining the transaminase polypeptide from the culture.

12. A transaminase catalyst obtainable from the method of claim 11, wherein the transaminase catalyst comprises cells or culture fluid containing the transaminase polypeptides obtained from the culture, or an article processed therewith, wherein the article refers to an extract obtained from the host cell, an isolated product obtained by isolating or purifying a transaminase from the extract, or an immobilized product obtained by immobilizing the host cell, an extract thereof, or isolated product of the extract.

13. A process for the preparing a compound of structural formula I, 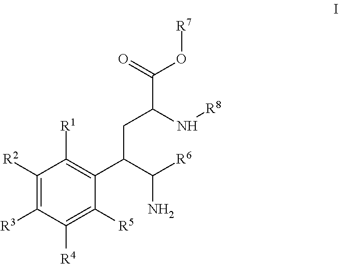
wherein the groups R¹, R², R³, R⁴, R⁵, can be optionally substituted or unsubstituted —H, C₁-C₆ hydrocarbon group, halogen (e.g. —F, —Cl, —Br, —I), —NO₂, —NO—NO, —SO₂R′ or —SOR′, —SR′, —NR′R′, —OR′, —CO₂R′ or —COR′, —C(O)NR′—C(O)NR′, —SO₂NH₂ or —SONH₂, —CN, CF₃; R⁶ can be a C₁-C₆ hydrocarbon group, C₁-C₆ haloalkyl, C₁-C₆ hydroxy-substituted hydrocarbon; R⁷ can be C₁-C₆ hydrocarbon group, C₁-C₆ haloalkyl, C₁-C₆ hydroxy-substituted hydrocarbon; R¹ can be CBZ protecting group, BOC protecting group, Fomc protecting group, Bn protecting group, methyl(ethyl)oxycarbonyl protecting group; wherein each R′ is independently selected from —H or C₁-C₄ hydrocarbon group; the process comprises, the substrate material of structural formula XI 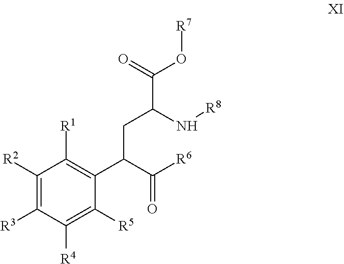
is contacted with the engineered polypeptide of claim 1.

14. The process of claim 13, wherein the product of structural formula I consists of one or more of the isomers shown as structural formulae II-VI, 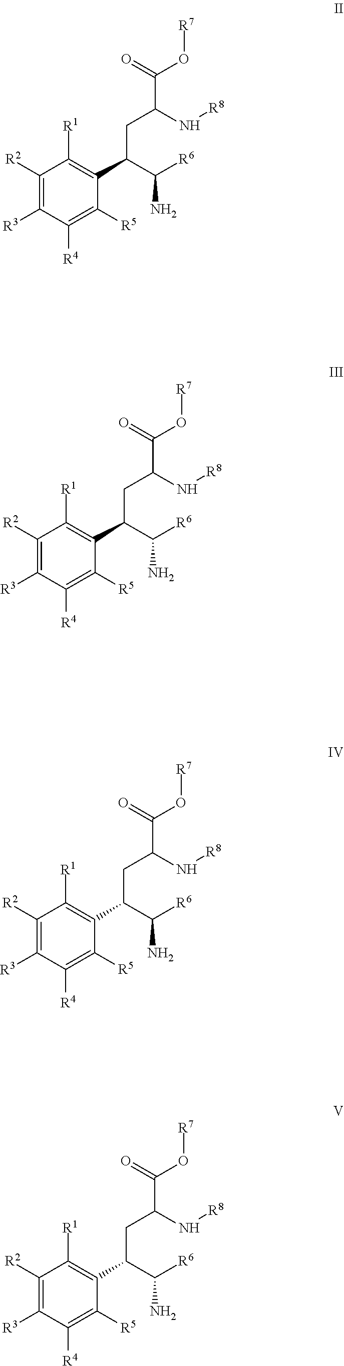
wherein under suitable reaction conditions, such as suitable temperature, pH and solvent conditions, some amine products shown as structural formula I can spontaneously form a ring to produce a lactam of structural formula VI: 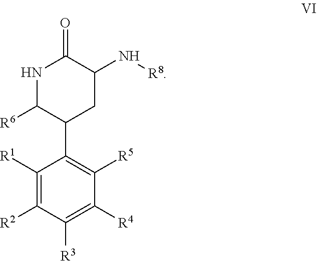
the compound shown as structural formula VI may consist of one or more isomers shown as structural formula VII-X:

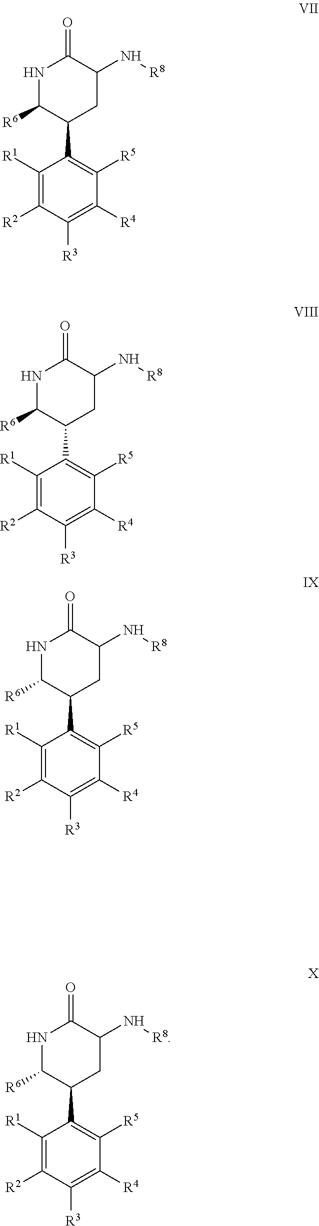

15. A process of preparing compounds of structural formula I1, 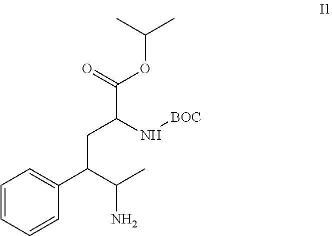 [SMILES: CC(C)OC(=O)C(CC(c1ccccc1)C(C)N)NC(=O)OC(C)(C)C]
wherein the process comprises, under suitable reaction conditions, the substrate material of structural formula S1 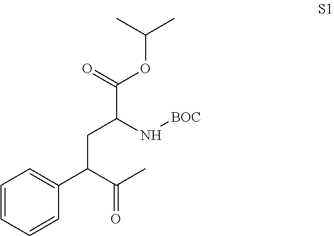 [SMILES: CC(=O)C(CC(NC(=O)OC(C)(C)C)C(=O)OC(C)C)c1ccccc1]
is contacted with the engineered polypeptide of claim 1.

16. A process of preparing a compound of structural formula L1, 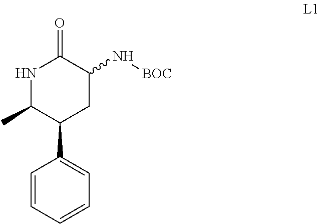 [SMILES: C[C@H]1NC(=O)C(NC(=O)OC(C)(C)C)C[C@H]1c1ccccc1]
wherein the process comprises, under suitable reaction conditions, the substrate material of structural formula S1, 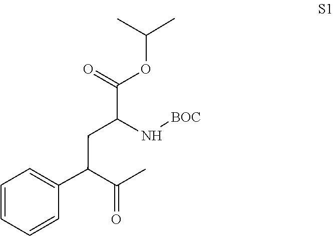 [SMILES: CC(=O)C(CC(NC(=O)OC(C)(C)C)C(=O)OC(C)C)c1ccccc1]
is contacted with the engineered polypeptide of claim 1.

17. The process of claim 16, wherein the dr value of the product compound of structural formula L1 (i.e. [T1+T2]/[D1+D2]) is at least 1, 2, 3, 5, 10, 20, 30, 40, 50, 60, 70, 80, 90, 100 or more.

18. The process as claimed in claim 13, wherein said reaction solvent or cosolvent comprises methanol, dimethyl sulfoxide (DMSO), acetonitrile (ACN), dimethyl formamide (DMF), methyl tert-butyl ether (MTBE), isopropyl acetate, ethanol, propanol, or isopropyl alcohol (IPA), or a mixture of 2 or more of them.

19. The process of claim 13, wherein said reaction conditions comprise a temperature of 10° C. to 65° C.

20. The process of claim 13, wherein said reaction conditions comprise pH 7.0 to pH 11.5.

21. The process of claim 13, wherein said substrate is present in a carrier amount of 10 g/L to 100 g/L.
